(19) United States
(12) Patent Application Publication
(10) Pub. No.: US 2010/0059261 A1
(43) Pub. Date: Mar. 11, 2010

(54) REACTIVE MONOMER AND RESIN COMPOSITION CONTAINING SAME

(75) Inventors: Hisashi Watanabe, Chiba (JP); Kenji Nakajima, Chiba (JP); Keizo Tanaka, Chiba (JP); Satoru Nanba, Chiba (JP)

Correspondence Address: OSHA LIANG L.L.P., TWO HOUSTON CENTER, 909 FANNIN, SUITE 3500, HOUSTON, TX, 77010, US

(73) Assignees: AMT LABORATORY CO., LTD., Yokkaichi-shi, Mie (JP); MANAC INC., Fukuyama-shi, Hiroshima (JP)

(21) Appl. No.: 11/993,457

(22) PCT Filed: Jun. 20, 2006
(86) PCT No.: PCT/JP2006/312270

§ 371 Date: Dec. 20, 2007

(30) Foreign Application Priority Data
Jun. 20, 2005 (JP) 2005-179773

(51) Int. Cl. H05K 1/00; C07D 209/48; C07D 307/77; C08K 5/3447; B32B 15/08; B32B 27/18; B32B 27/36

(52) U.S. Cl. 174/258; 548/476; 549/303; 524/94; 428/458; 428/337; 428/412; 428/473.5

(57) ABSTRACT

A material for use in flexible printed wiring board having high reliability and allowing for processing fine wiring includes a compound represented by the following general formula (I):

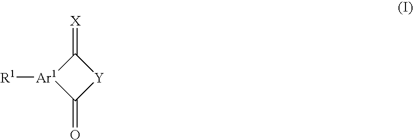

wherein one of X and Y represents O and the other represents NAr²R², and R¹, R², Ar¹, and Ar² are as defined in the specification, and a resin composition comprising the compound as a reactive monomer.

### FIELD OF THE INVENTION

The present invention relates to a novel compound useful as a reactive monomer. In addition, the present invention is also concerned with a resin composition comprising the compound useful as a reactive monomer, and a heat-resistant adhesive agent comprising the resin composition. Further, the present invention is also concerned with a metal laminate and an aromatic polymer laminate, which are obtained using the above adhesive agent, and which are materials for use in, e.g., flexible printed wiring board having high reliability and allowed to process fine wiring.

### BACKGROUND ART

In recent years, as electronic devices, such as flat panel displays, are improved in function and reduced in thickness, electronic parts and boards mounted on the electronic devices are required to have improved function, higher performance, and higher density. Further, it is considered that a wafer become highly integrated for improving the yield and function and increasing pixels. Therefore, a COF (chip on film) method is used for bonding a driver IC and a flexible board, which is advantageous to fine pitch wiring, instead of a TAB (tape automated bonding) method. A COF is obtained by forming a copper wiring pattern by etching on a flexible copper clad laminate in which a copper foil or the like is stuck on a resin film comprised of, e.g., polyimide, and then mounting an IC chip on the resultant laminate through a gold bump.

Generally, a flexible copper clad laminate is classified into a three-layer copper clad laminate comprising a copper foil and a polyimide film which are stuck together with an adhesive agent such as an epoxy- or acrylic-based adhesive agent, and a two-layer copper clad laminate comprising a polyimide film and a copper foil which are unified without using an adhesive agent such as an epoxy- or acrylic-based adhesive agent. In the COF, a two-layer copper clad laminate is used as a flexible copper clad laminate which serves as a substrate, and further, reduction of the copper foil in thickness is essential to form fine wiring having a line/space pitch of 25 μm/25 μm or smaller.

As examples of methods for producing a two-layer copper clad laminate, there can be mentioned a metallizing method, a casting method, and a laminating method. The metallizing method is a method in which a thin film of a metal, such as Cr, is deposited on a polyimide film by, e.g., sputtering, and a copper film having a predetermined thickness is formed on the resultant film by sputtering or plating, but the adhesion to copper is poor or cracks occur due to the metal, e.g., Cr, and hence the reliability is unsatisfactory (see, for example, Japanese Unexamined Patent Publication No. 2002-172734). The casting method is a method in which a polyimide varnish or a varnish comprising polyamic acid which is a precursor of polyimide is applied to a copper foil and cured by heating to form a polyimide film on the copper foil, and thus provides a copper clad laminate having high adhesivity to copper. However, a polyimide layer having an uneven thickness is frequently formed, causing a defective product. Further, the step for applying a varnish to a copper foil technically restricts the reduction of the copper foil in thickness (see, for example, Japanese Unexamined Patent Publication No. Sho 62-212140). The laminating method is a method in which a copper foil and a polyimide film are laminated through thermoplastic polyimide by pressing them, and thus provides a laminate having a uniform thickness. However, for achieving thermal adhesiveness of the film, the lamination temperature must be the glass transition temperature of the thermoplastic polyimide or higher (generally 250° C. or higher, which varies depending on the thermoplastic polyimide used). Further, in such a high temperature region, wrinkles occur due to a difference in dimensional change rate between the substrates laminated, leading to problems of bad appearance, poor insulation, and poor conduction. In addition, the adhesive layer is thermoplastic and hence, when an IC is mounted on the laminate, a problem occurs in that the mounted parts sink (see, for example, Japanese Patent Publication No. 2004-188962).

On the other hand, as a thermosetting resin, an imide oligomer having a phenylethynyl skeleton at the end has been reported, but the imide oligomer before being cured by heating has a glass transition temperature of 208 to 262° C., and thus it is difficult to achieve thermal adhesiveness of this oligomer in a temperature region of 200° C. or lower (see, for example, U.S. Pat. No. 5,567,800). Further, as a thermosetting adhesive agent, a polyimide resin composition comprising a mixture of aromatic polyimide and polyimide having a phenylethynyl group at the end has been reported. Specifically, it has been reported that a phenylethynyl-terminated imide oligomer is mixed into soluble polyimide modified with silicon, obtaining an adhesive agent having improved heat resistance and improved adhesive properties. However, this resin composition before being cured has a glass transition temperature of 216° C., and the resin composition after being cured has a glass transition temperature of 228° C., that is, the resin composition has a small difference in glass transition temperature between before and after being cured, and hence has poor processability and poor heat resistance (see, for example, Japanese Unexamined Patent Publication No. 2003-213130).

### DISCLOSURE OF THE INVENTION

### Problems to be Solved by the Invention

An object of the present invention is to solve the above problems accompanying the prior art and to provide a novel compound suitable for a material constituting, e.g., a COF. Further, another object of the present invention is to provide a resin composition comprising the compound as a reactive monomer and a heat-resistant adhesive agent comprising the resin composition. Still another object of the present invention is to provide a metal laminate and an aromatic polymer laminate obtained using the above adhesive agent, and these are useful as a material for a flexible printed wiring board which is allowed to process fine wiring.

### Means to Solve the Problems

The present invention is directed to a compound represented by the following general formula (I):

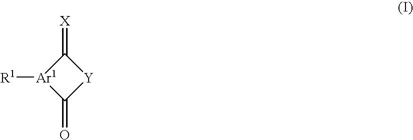

wherein:
one of X and Y represents O and the other represents NAr²R²;
R¹ and R², which may be the same or different, represent hydrogen or an organic group having 2 to 36 carbon atoms and containing at least one carbon-carbon double bond or carbon-carbon triple bond, with the proviso that R¹ and R² are not simultaneously hydrogen;
Ar¹ represents an organic group having 6 to 36 carbon atoms; and
Ar² represents an organic group having 6 to 36 carbon atoms.

In addition, the present invention is directed to a resin composition comprising the compound as a reactive monomer, and a heat-resistant adhesive agent comprising the resin composition. Further, the present invention is directed to a metal laminate comprising an aromatic polymer and a metallic foil laminated through the heat-resistant adhesive agent, and an aromatic polymer laminate.

### EFFECT OF THE INVENTION

By using as an adhesive agent layer a resin composition comprising the compound of the present invention as a reactive monomer, an insulating film comprised of an aromatic polymer, such as polyimide, can be laminated onto a metallic foil, particularly a copper foil, at a temperature even lower than the lamination temperature employed when using a conventional adhesive agent comprised of thermoplastic polyimide. Further, with respect to the metal laminate for COF, not only can reliability including heat resistance, adhesive properties, and electrical properties be improved, but also the problems of bad appearance including the occurrence of wrinkles due to a difference in dimensional change rate can be remarkably prevented, so that the productivity can be dramatically improved, thus enabling inexpensive and efficient

### BRIEF DESCRIPTION OF THE DRAWINGS

FIG. 1 is a ¹H-NMR chart of the compound obtained in Example 1.

FIG. 2 is an IR chart of the compound obtained in Example 1.

FIG. 3 is a ¹H-NMR chart of the compound obtained in Example 3.

FIG. 4 is an IR chart of the compound obtained in Example 3.

FIG. 5 is a ¹H-NMR chart of the compound obtained in Example 4.

FIG. 6 is an IR chart of the compound obtained in Example 4.

### BEST MODE FOR CARRYING OUT THE INVENTION

The compound of the present invention is first described. The compound of the present invention is a compound represented by the following general formula (I):

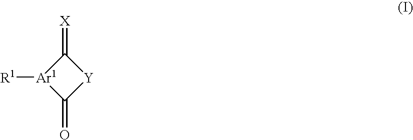

wherein:
one of X and Y represents O and the other represents NAr²R²;
R¹ and R², which may be the same or different, represent hydrogen or an organic group having 2 to 36 carbon atoms and containing at least one carbon-carbon double bond or carbon-carbon triple bond, with the proviso that R¹ and R² are not simultaneously hydrogen;
Ar¹ represents an organic group having 6 to 36 carbon atoms; and
Ar² represents an organic group having 6 to 36 carbon atoms.

Specifically, the compound of general formula (I) of the present invention is an imide compound or an isoimide compound which is a regioisomer thereof represented by the following general formula (1) or (2):

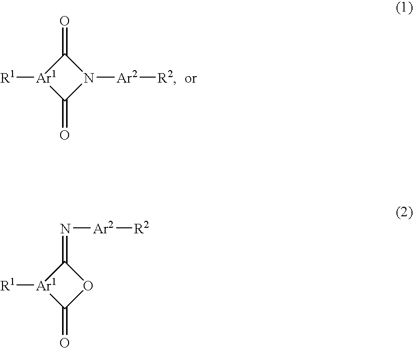

wherein R¹, R², Ar¹, and Ar² are as defined above.

Specifically, in the compound of general formula (I) of the present invention, R¹ is preferably a group represented by the following formula (3):

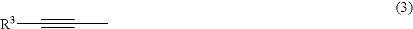

wherein R³ represents hydrogen or an organic group having 1 to 34 carbon atoms, especially hydrogen, a C₆-C₁₈ aryl group, or a group represented by the following formula:

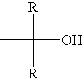

wherein each R independently represents hydrogen, a C₁-C₄ alkyl group, or a C₆-C₁₈ aryl group.

More specifically, in the compound of general formula (I) of the present invention, R³ especially preferably represents hydrogen, phenyl, or a group represented by the following formula:

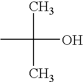

Alternatively, in the compound of general formula (I) of the present invention, R² is preferably a group represented by the following formula (4):

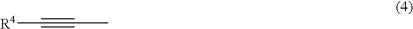

wherein R⁴ represents hydrogen or an organic group having 1 to 34 carbon atoms, especially hydrogen, a C₆-C₁₈ aryl group, or a group represented by the following formula:

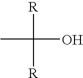

wherein each R independently represents hydrogen, a C₃-C₄ alkyl group, or a C₆-C₁₈ aryl group.

More specifically, in the compound of general formula (I) of the present invention, R⁴ especially preferably represents hydrogen, phenyl, or a group represented by the following formula:

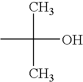

Further specifically, the compound of the present invention is preferably the compound wherein R¹ and R², which may be the same or different, are selected from ethynyl, phenylethynyl, and a group represented by the following formula:

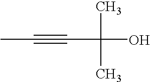

further in these cases, especially preferred is the compound wherein Ar¹ is benzenetriyl and Ar² is phenylene.

In the preparation of these compounds, generally, a dicarboxylic anhydride component and an amine component are first reacted with each other to prepare a corresponding amic acid. With respect to the preparation of an auric acid, there is no particular limitation, and the preparation can be conducted by a known method, and is generally conducted in a solvent.

Examples of dicarboxylic anhydride components used in the preparation of the compound of the present invention include compounds represented by the following general formula (II):

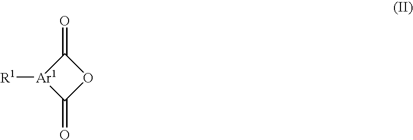

wherein R¹ represents hydrogen or an organic group having 2 to 36 carbon atoms and containing at least one carbon-carbon double bond or carbon-carbon triple bond, and Ar¹ represents an organic group having 6 to 36 carbon atoms.

In R¹ of the general formulas (I) and (II), the organic group having 2 to 36 carbon atoms and containing at least one carbon-carbon double bond or carbon-carbon triple bond is, for example, a C₂-C₃₆ alkenyl group, a C₂-C₃₅ alkynyl group, a C₆-C₃₄ aryl-C₂-C₃₀ alkenyl group, a C₂-C₃₀ alkenyl-C₆-C₃₄ aryl group, a C₆-C₃₄ aryl-C₂-C₃₀ alkynyl group, or a C₂-C₃₀ alkynyl-C₆-C₃₄ aryl group, preferably a C₂-C₃₆ alkynyl group or a C₆-C₃₄ aryl-C₂-C₃₀ alkynyl group, further preferably a C₂-C₆ alkynyl group or a C₆-C₁₆ aryl-C₂-C₆ alkynyl group, especially a C₂-C₆ alkynyl group or C₆-C₁₈ aryl-C₂-C₆ alkynyl group optionally substituted by a hydroxyl group, specifically, ethynyl, phenylethynyl, or a group of the following formula:

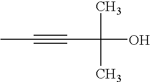

Therefore, when R¹ is a group represented by the formula (3), in R³ of the formula (3), the organic group having 1 to 34 carbon atoms is, for example, a C₁-C₃₄ alkyl group, a C₆-C₃₄ aryl group, a C₁-C₂₈ alkyl-C₆-C₃₃ aryl group, or a C₆-C₃₃ aryl-C₁-C₂₈ alkyl group, preferably a C₁-C₃₄ alkyl group, a C₆-C₃₄ aryl-C₂-C₃₀ alkynyl group, or a C₆-C₃₄ aryl group, further preferably a C₁-C₄ alkyl group, a C₆-C₁₈ aryl-C₁-C₄ alkyl group, or a C₆-C₁₈ aryl group, especially a C₁-C₄ alkyl group or C₆-C₁₈ aryl-C₁-C₄ alkyl group optionally substituted by a hydroxyl group at the α-position, for example, a group represented by the following formula:

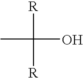

wherein each R independently represents hydrogen, a C₁-C₄ alkyl group, or a C₆-C₁₈ aryl group,
   or a C₅-C₁₈ aryl group, specifically, phenyl or a group represented by the following formula:

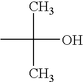

In Ar¹ of the general formulas (I) and (II), the organic group having 6 to 36 carbon atoms is a trivalent group of, for example, a monocyclic or condensed polycyclic compound having 6 to 36 carbon atoms or a non-condensed polycyclic aromatic compound comprising the monocyclic or condensed polycyclic compounds bonded to one another directly or through a bridging linkage (wherein the bridging linkage may be, for example, —O—, —CO—, —COO—, —NH—, alkylene, sulfinyl, sulfonyl, or a combination thereof, and these compounds and bridging linkages may be optionally substituted by one or more halogens, hydroxyls, or alkyl groups, alkenyl groups, alkynyl groups, halogenated alkyl groups or alkoxy groups having 1 to 6 carbon atoms), preferably a trivalent group selected from the following formulas:

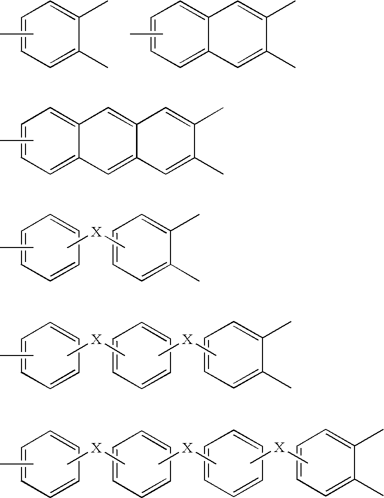

wherein X, which may be the same or different, represents a single bond, —O—, —CO—, —CH₂—, —C(CH₃)₂— or —C(CF₃)₂—.

Specific examples of dicarboxylic anhydrides represented by the general formula (II) include phthalic anhydride, naphthalenedicarboxylic anhydride, anthracenedicarboxylic anhydride, 4-ethynylphthalic anhydride, 3-ethynylphthalic anhydride, 4-phenylethynylphthalic anhydride, 3-phenylethynylphthalic anhydride, 4-(3-hydroxy-3-methyl-1-but-1-ynyl)phthalic anhydride, 4-(3-hydroxy-3-methyl-1-but-1-ynyl)phthalic anhydride, ethynylnaphthalenedicarboxylic anhydride, phenylethynylnaphthalenedicarboxylic anhydride, ethynylanthracenedicarboxylic anhydride, phenylethynylanthracenedicarboxylic anhydride, 4-naphthylethynylphthalic anhydride, 3-naphthylethynylphthalic anhydride, naphthylethynylnaphthalenedicarboxylic anhydride, naphthylethynylanthracenedicarboxylic anhydride, 4-anthracenylethynylphthalic anhydride, 3-anthracenylethynylphthalic anhydride, anthracenylethynylnaphthalenedicarboxylic anhydride, anthracenylethynylanthracenedicarboxylic anhydride, biphenyl-3,4-dicarboxylic anhydride, 3′-ethynylbiphenyl-3,4-dicarboxylic anhydride, 4′-ethynylbiphenyl-3,4-dicarboxylic anhydride, 3′-phenylethynylbiphenyl-3,4-dicarboxylic anhydride, 4′-phenylethynylbiphenyl-3,4-dicarboxylic anhydride, diphenylether-3,4-dicarboxylic anhydride, 3′-ethynyldiphenylether-3,4-dicarboxylic anhydride, 4′-ethynyldiphenylether-3,4-dicarboxylic anhydride, 3′-phenylethynyldiphenylether-3,4-dicarboxylic anhydride, 4′-phenylethynyldiphenylether-3,4-dicarboxylic anhydride, benzophenone-3,4-dicarboxylic anhydride, 3′-ethynylbenzophenone-3,4-dicarboxylic anhydride, 4′-ethynylbenzophenone-3,4-dicarboxylic anhydride, 3′-phenylethynylbenzophenone-3,4-dicarboxylic anhydride, 4′-phenylethynylbenzophenone-3,4-dicarboxylic anhydride and the like. In these anhydrides, the hydrogen atom on the aromatic ring can be replaced by an alkyl group, alkenyl group, alkynyl group or alkoxy group having 1 to 6 carbon atoms or a halogen atom. The compound of general formula (I) of the present invention contains at least one carbon-carbon double bond or triple bond, and the dicarboxylic anhydride is appropriately selected depending on the amine component used, and but, from the viewpoint of easy availability, it is desirable to use 4-phenylethynylphthalic anhydride, 4-ethynylphthalic anhydride, or 4-(3-hydroxy-3-methyl-1-but-1-ynyl)phthalic anhydride. 4-Phenylethynylphthalic anhydride can be prepared by a method described in, for example, Japanese Unexamined Patent Publication No. 2003-73372, and 4-ethynylphthalic anhydride and 4-(3-hydroxy-3-methyl-1-but-1-ynyl)phthalic anhydride can be prepared by a method described in, for example, Japanese Unexamined Patent Publication No Hei 10-114691 or Japanese Unexamined Patent Publication No. 2004-123573. The above two or more anhydrides can be used in combination.

On the other hand, examples of amine components used in the preparation of the compound of the present invention include compounds represented by the following general formula (III):

H₂N—Ar²—R² (III)
wherein R² represents hydrogen or an organic group having 2 to 36 carbon atoms and containing at least one carbon-carbon double bond or carbon-carbon triple bond, and Ar² represents an organic group having 6 to 36 carbon atoms.

In R² of the general formulas (I) and (III), the organic group having 2 to 36 carbon atoms and containing at least one carbon-carbon double bond or carbon-carbon triple bond is, for example, a C₂-C₃₆ alkenyl group, a C₂-C₃₆ alkynyl group, a C₆-C₃₄ aryl-C₂-C₃₀ alkenyl group, a C₂-C₃₀ alkenyl-C₆-C₃₄ aryl group, a C₆-C₃₄ aryl-C₂-C₃₀ alkynyl group, or a C₂-C₃₀ alkynyl-C₆-C₃₄ aryl group, preferably a C₂-C₃₆ alkynyl group or a C₆-C₃₄ aryl-C₂-C₃₀ alkynyl group, further preferably a C₂-C₆ alkynyl group or a C₆-C₁₈ aryl-C₂-C₆ alkynyl group, especially a C₂-C₆ alkynyl group or C₆-C₁₈ aryl-C₂-C₆ alkynyl group optionally substituted by a hydroxyl group at the α-position, specifically, ethynyl, phenylethynyl, or a group of the following formula:

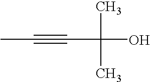

Therefore, when R² is a group represented by the formula (4), in R⁴ of the formula (4), the organic group having 1 to 34 carbon atoms is, for example, a C₁-C₃₄ alkyl group, a C₆-C₃₄ aryl group, a C₁-C₂₈ alkyl-C₆-C₃₃ aryl group, or a C₆-C₃₃ aryl-C₁-C₂₈ alkyl group, preferably a C₁-C₃₄ alkyl group, a C₆-C₃₄ aryl-C₂-C₃₀ alkynyl group, or a C₆-C₃₄ aryl group, further preferably a C₁-C₄ alkyl group, a C₆-C₁₈ aryl-C₁-C₄ alkyl group, or a C₆-C₁₈ aryl group, especially a C₁-C₄ alkyl group or C₆-C₁₈ aryl-C₁-C₄ alkyl group optionally substituted by a hydroxyl group at the α-position, for example, a group represented by the following formula:

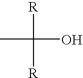

wherein each R independently represents hydrogen, a C₁-C₄ alkyl group, or a C₆-C₁₈ aryl group,
   or a C₆-C₁₈ aryl group, specifically, phenyl or a group represented by the following formula:

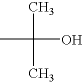

In Ar² of the general formulas (I) and (III), the organic group having 6 to 36 carbon atoms is a divalent group of, for example, a monocyclic or condensed polycyclic compound having 6 to 36 carbon atoms or a non-condensed polycyclic aromatic compound comprising the monocyclic or condensed polycyclic compounds bonded to one another directly or through a bridging linkage (wherein the bridging linkage may be, for example, —O—, —CO—, —COO—, —NH—, alkylene, sulfinyl, sulfonyl, or a combination thereof, and these compounds and bridging linkages may be optionally substituted by one or more halogens, hydroxyls, or alkyl groups, alkenyl groups, alkynyl groups, halogenated alkyl groups or alkoxy groups having 1 to 6 carbon atoms), preferably a divalent group selected from the following formulas:

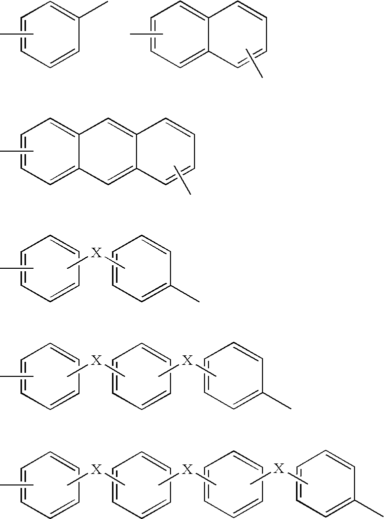

wherein X, which may be the same or different, represents a single bond, —O—, —CO—, —CH₂—, —C(CH₃)₂—, or —C(CF₃)₂—.

Specific examples of amine components represented by the general formula (III) include aniline, o-toluidine, m-toluidine, p-toluidine, 2,3-xylidine, 3,4-xylidine, 1-naphthylamine, 2-naphthylamine, 1-aminoanthracene, 2-aminoanthracene, 9-aminoanthracene, 3-phenoxyaniline, 4-phenoxyaniline, 3-aminobenzophenone, 4-aminobenzophenone, 3-aminophenylacetylene, 4-aminophenylacetylene, 3-phenylethynylaniline, 4-phenylethynylaniline, 4-(3-hydroxy-3-methyl-1-but-1-ynyl)aniline, 4-(3-hydroxy-3-methyl-1-but-1-ynyl)aniline, 3-naphthylethynylaniline, 4-naphthylethynylaniline, 3-anthracenylethynylaniline, 4-anthracenylethynylaniline and the like. In these amine components, the hydrogen atom on the aromatic ring can be replaced by an alkyl group, alkenyl group, alkynyl group or alkoxy group having 1 to 6 carbon atoms or a halogen atom. The compound of general formula (I) of the present invention contains at least one carbon-carbon double bond or triple bond, and the amine component is appropriately selected depending on the dicarboxylic anhydride used, and but, from the viewpoint of easy availability, it is desirable to use 3-aminophenylacetylene, 4-aminophenylacetylene, 3-phenylethynylaniline, 4-phenylethynylaniline, 4-(3-hydroxy-3-methyl-1-but-1-ynyl)aniline, or 3-(3-hydroxy-3-methyl-1-but-1-ynyl)aniline. 3-Aminophenylacetylene can be prepared by a method described in, for example, Japanese Unexamined Patent Publication No. Hei 10-36325, 4-aminophenylacetylene can be prepared by a method described in, for example, Japanese Unexamined Patent Publication No. Hei 9-143129, and 4-(3-hydroxy-3-methyl-1-but-1-ynyl)aniline can be prepared by a method described in, for example, Japanese Unexamined Patent Publication No. Hei 10-114691. The above two or more amine components can be used in combination.

With respect to the solvent used in the reaction of auric acid, there is no particular limitation as long as it is an inert solvent in the reaction, and, for example, N,N-dimethylformamide, N,N-dimethylacetamide, N-methyl-2-pyrrolidone, dimethyl sulfoxide, tetramethylurea, tetrahydrofuran or the like can be used individually or in the form of a mixed solvent. Especially preferred is N,N-dimethylacetamide, N-methyl-2-pyrrolidone, or tetrahydrofuran. A solvent, such as benzene, toluene, xylene, mesitylene, chlorobenzene, diglyme, or triglyme, can be mixed into the above solvent in an arbitrary amount. The reaction is generally conducted at a solute concentration of 5 to 80%.

Then, the amic acid obtained is imidized or isoimidized. The imidization reaction is conducted by dehydrating the amic acid obtained in the above reaction by a known method. For example, in a chemical imidization method, the amic acid solution is subjected to dehydration by adding a dehydrating agent such as, but not limited to, acetic anhydride, trifluoroacetic anhydride, polyphosphoric acid, phosphorus pentaoxide, phosphorus pentachloride, or thionyl chloride, or a mixture thereof. A catalyst, such as pyridine, can be used. In a thermal imidization method, the amic acid solution obtained in the above reaction is subjected to dehydration by mixing a solvent, such as benzene, toluene, xylene, mesitylene, chlorobenzene, diglyme, or triglyme, in an arbitrary amount into the amic acid solution and heating the resultant mixture while removing water formed by ring closure from the reaction system. These solvents can be used individually or in combination. On the other hand, the isoimidization reaction is conducted by dehydrating the amic acid obtained in the above reaction by a known method. For example, the amic acid is subjected to dehydration by adding a dehydrating agent, such as trifluoroacetic anhydride or N,N-dicyclohexylcarbodiimide, or a mixture thereof. A catalyst, such as pyridine, can be used.

The “isoimide” corresponds to a regioisomer of an imide, and has a structure represented by the following formula:

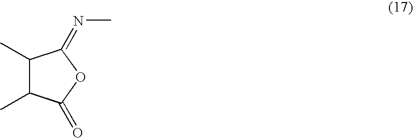

in the molecule thereof, and the isoimide undergoes intramolecular rearrangement at a temperature of 200 to 300° C. and changes to an imide.

The compound of general formula (I) of the present invention can be used either in the form of powder obtained by pouring the reaction mixture obtained after the completion of imidization or isoimidization into a solvent, such as water or alcohol, to effect reprecipitation, and collecting the resultant crystals by filtration and drying them, or in the form of a solution obtained merely by removing by-products of an isoimidizing agent, such as dicyclohexylurea, by filtration from the reaction mixture.

With respect to the compound of the present invention, compounds especially preferred as reactive monomers are compounds represented by the following formulas (5) to (12):

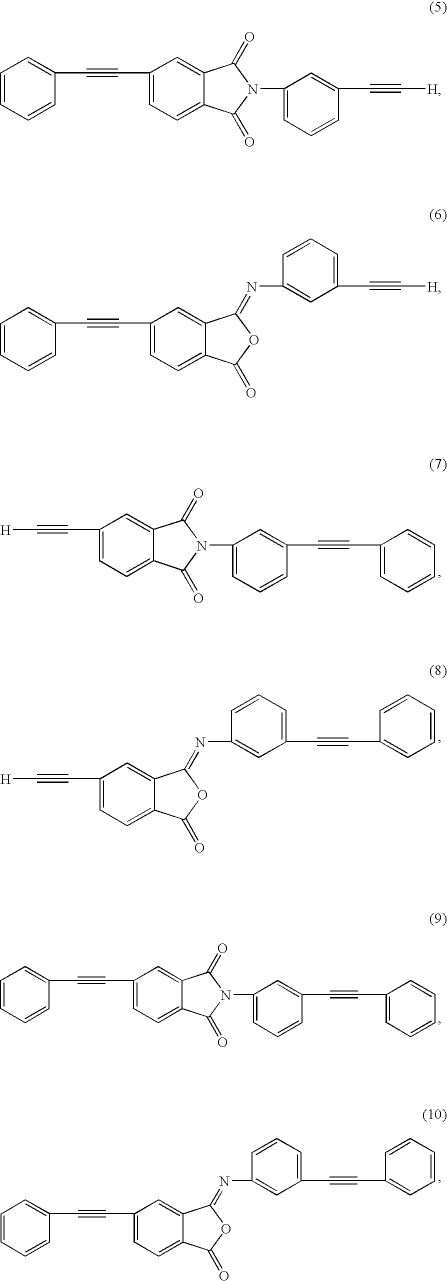

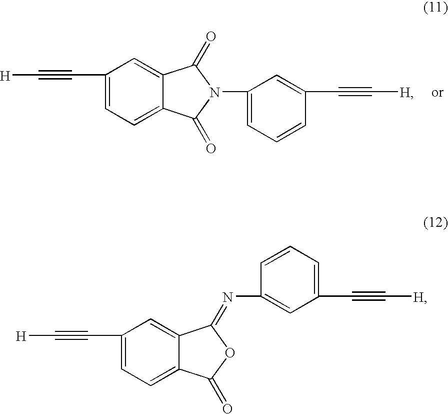

and these compounds can be either individually produced as an imide compound or isoimide compound and used as a reactive monomer, or produced in the form of a mixture of isomers and used as a reactive monomer.

Further, with respect to the compound of the present invention, other compounds preferred as reactive monomers are compounds represented by the following formulas (13) to (17):

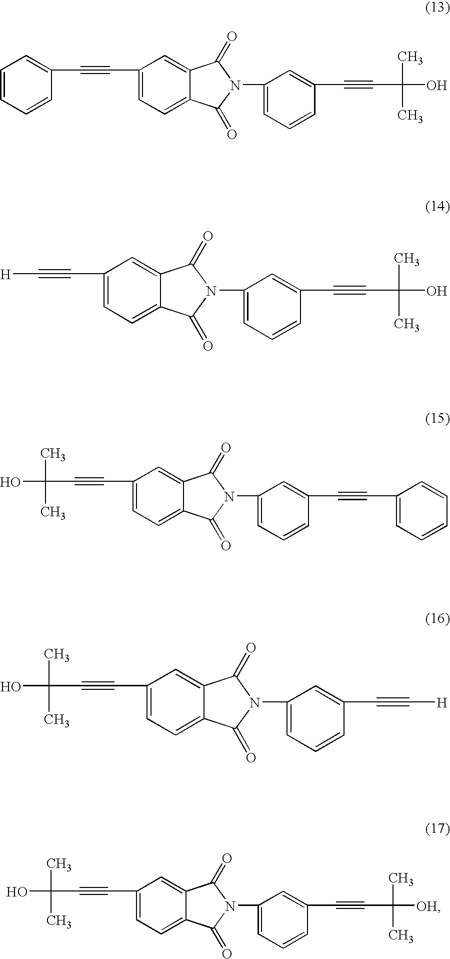

and these compounds can be either individually produced as an imide compound or isoimide compound and used as a reactive monomer, or produced in the form of a mixture of isomers and used as a reactive monomer.

The resin composition of the present invention comprises (a) polyimide or (a′) polyamic acid, and (b) the above-obtained compound of general formula (I) of the present invention. The resin composition of the present invention preferably contains component (a) or (a′) and component (b) in a weight ratio of 99/1 to 40/60, especially preferably in a weight ratio of 95/5 to 50/50. For further improving the heat resistance, adhesive properties, and the like, the resin composition of the present invention can be prepared by mixing (c) a thermosetting resin having a crosslinkable group into the above resin composition. The latter resin composition preferably contains component (a) or (a′) and component (c) in a weight ratio of 95/5 to 5/95, especially preferably in a weight ratio of 80/20 to 20/80. Further, in the resin composition containing the components in the above weight ratio, the ratio of the total weight of components (a) or (a′) and (c) to the weight of compound (b) of general formula (I) of the present invention is preferably 99/1 to 40/60, especially preferably 95/5 to 50/50.

Polyimide (a) and/or polyamic acid (a′) is first described. The polyimide and/or polyamic acid used in the resin composition of the present invention is represented by the following general formula (18):

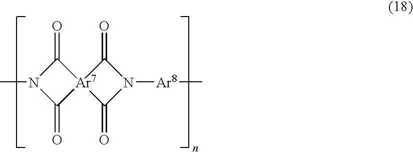

or the following general formula (19):

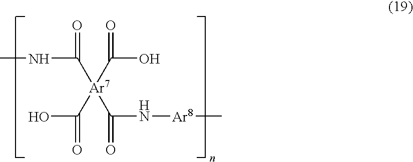

wherein n represents a number of 20 or more; Ar⁷ represents a tetracarboxylic acid residue; and Ar⁸ represents a diamine residue.

With respect to the preparation of the polyimide and/or polyamic acid, there is no particular limitation, and the preparation can be conducted by a known method, and is generally conducted in a solvent. The polyimide and/or polyamic acid is prepared by reacting an aromatic tetracarboxylic dianhydride with an aromatic diamine in a polar solvent. Specific examples of the tetracarboxylic dianhydrides used in the preparation (which produce tetracarboxylic acid residue Ar⁷) include pyromellitic dianhydride, 3,3′,4,4′-biphenyltetracarboxylic dianhydride, 2,3′,3,4′-biphenyltetracarboxylic dianhydride, 2,2′,3,3′-biphenyltetracarboxylic dianhydride, 4,4′-oxydiphthalic dianhydride, 3,4′-oxydiphthalic dianhydride, 3,3′-oxydiphthalic dianhydride, 3,3′,4,4′-benzophenonetetracarboxylic dianhydride, 3,3′,4,4′-diphenyl sulfone tetracarboxylic dianhydride, 2,2-bis(3,4-dicarboxyphenyl)propane dianhydride, 2,2-bis(3,4-dicarboxyphenyl)hexafluoropropane dianhydride, and 1,2,7,8-naphthalenetetracarboxylic dianhydride. It is desired that the polyimide and/or polyamic acid represented by the general formula (18) or (19) has high affinity to a copper foil and polyimide, and therefore, the tetracarboxylic dianhydride used varies depending on the desired molecular weight or the type of the diamine selected, but it is desirable to use pyromellitic dianhydride, 4,4′-oxydiphthalic dianhydride, 3,3′,4,4′-biphenyltetracarboxylic dianhydride, 3,3′,4,4′-benzophenonetetracarboxylic dianhydride, 3,3′,4,4′-diphenyl sulfone tetracarboxylic dianhydride, 2,2-bis(3,4-dicarboxyphenyl)propane dianhydride, or 2,2-bis(3,4-dicarboxyphenyl)hexafluoropropane dianhydride. The above two or more dianhydrides can be used in combination.

Examples of aromatic diamines (which produce diamine residue Ar⁸) include aromatic diamines having one aromatic group, such as p-phenylenediamine, m-phenylenediamine, p-aminobenzylamine, m-aminobenzylamine, diaminotoluenes, diaminoxylenes, diaminonaphthalenes, and diaminoanthracenes; aromatic diamines having two aromatic groups, such as 4,4′-diaminobiphenyl, 3,4′-diaminobiphenyl, 3,3′-diaminobiphenyl, o-tolidine, m-tolidine, o-dianisidine, 4,4′-diaminodiphenylmethane, 3,4′-diaminodiphenylmethane, 3,3′-diaminodiphenylmethane, 4,4′-diaminodiphenyl ether, 3,4′-diaminodiphenyl ether, 3,3′-diaminodiphenyl ether, 4,4′-diaminodiphenyl sulfone, 3,4′-diaminodiphenyl sulfone, 3,3′-diaminodiphenyl sulfone, 4,4′-diaminodiphenyl ketone, 3,4′-diaminodiphenyl ketone, 3,3′-diaminodiphenyl ketone, 2,2-bis(4-aminophenoxy)propane, 2,2-bis(3-aminophenoxy)propane, and 2-(3-aminophenyl)-2-(4-aminophenyl)propane; aromatic diamines having three aromatic groups, such as 1,4-bis(4-aminophenoxy)benzene, 1,4-bis(3-aminophenoxy)benzene, 1,3-bis(4-aminophenoxy)benzene, 1,3-bis(3-aminophenoxy)benzene, 1,4-bis(4-aminobenzoyl)benzene, 1,4-bis(3-aminobenzoyl)benzene, 1,3-bis(4-aminobenzoyl)benzene, 1,3-bis(3-aminobenzoyl)benzene, and 9,9-bis(4-aminophenyl)fluorene; aromatic diamines having four or more aromatic groups, such as 2,2-bis[4-(4-aminophenoxy)phenyl]propane, 4,4′-bis(4-aminophenoxy)biphenyl, 4,4′-bis(3-aminophenoxy)biphenyl, bis[4-(4-aminophenoxy)phenyl]sulfone, bis[4-(3-aminophenoxy)phenyl]sulfone, bis[4-(4-aminophenoxy)phenyl]ether, bis[4-(3-aminophenoxy)phenyl]ether, 4,4′-bis(4-aminophenoxy)benzophenone, 4,4′-bis(3-aminophenoxy)benzophenone, 1,4-bis[4-(2-,3-, or 4-aminophenoxy)benzoyl]benzene, 1,3-bis[4-(2-,3-, or 4-aminophenoxy)benzoyl]benzene, 1,4-bis[3-(2-,3-, or 4-aminophenoxy)benzoyl]benzene, 1,3-bis[3-(2-,3-, or 4-aminophenoxy)benzoyl]benzene, 4,4′-bis[4-(2-,3-, or 4-aminophenoxy)benzoyl]diphenyl ether, 4,4′-bis[3-(2-,3-, or 4-aminophenoxy)benzoyl]diphenyl ether, 4,4′-bis[4-(2-, 3-, or 4-aminophenoxy)benzoyl]biphenyl, 4,4′-bis[3-(2-,3-, or 4-aminophenoxy)benzoyl]biphenyl, 4,4′-bis[4-(2-,3-, or 4-aminophenoxy)benzoyl]diphenyl sulfone, and 4,4′-bis[3-(2-, 3-, or 4-aminophenoxy)benzoyl]diphenyl sulfone. It is desired that the polyimide and/or polyamic acid represented by the general formula (18) or (19) has high affinity to a copper foil and polyimide, and therefore, the aromatic diamine used varies depending on the desired molecular weight or the type of the tetracarboxylic dianhydride selected, but, from the viewpoint of easy availability or the like, specifically, it is preferable to use p-phenylenediamine, m-phenylenediamine, 4,4′-diaminodiphenyl ether, 3,4′-diaminodiphenyl ether, 1,3-bis(4-aminophenoxy)benzene, 1,3-bis(3-aminophenoxy)benzene, 2,2-bis[4-(4-aminophenoxy)phenyl]propane, 4,4′-bis(4-aminophenoxy)biphenyl, 4,4′-bis(3-aminophenoxy)biphenyl, bis[4-(4-aminophenoxy)phenyl]sulfone, bis[4-(3-aminophenoxy)phenyl]sulfone, or 9,9-bis(4-aminophenyl)fluorene. The above two or more diamine compounds can be used in combination.

A siloxanediamine represented by the following general formula (20):

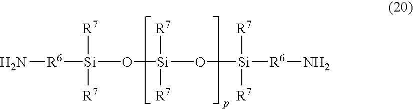

wherein p represents a mixed value of integers of 0 to 20; R⁷ represents a methyl group, an isopropyl group, a phenyl group, or a vinyl group; and R⁸ represents a hydrocarbon group having 1 to 7 carbon atoms, e.g., trimethylene, tetramethylene, or phenylene
   can be copolymerized together with the above compounds in an amount range of 1 to 50 mol %.

With respect to the solvent used in the reaction of polyimide and/or polyamic acid, there is no particular limitation as long as it is an inert solvent in the reaction, and, for example, N,N-dimethylformamide, N,N-dimethylacetamide, N-methyl-2-pyrrolidone, dimethyl sulfoxide, tetramethylurea, tetrahydrofuran or the like can be used individually or in the form of a mixed solvent. Especially preferred is N,N-dimethylacetamide or N-methyl-2-pyrrolidone. A solvent, such as benzene, toluene, xylene, mesitylene, chlorobenzene, diglyme, or triglyme, can be mixed into the above solvent in an arbitrary amount. The reaction is generally conducted at a solute concentration of 5 to 80%.

Then, the imidization reaction is conducted by dehydrating the polyamic acid obtained in the above reaction by a known method. For example, in a chemical imidization method, the polyamic acid solution is subjected to dehydration by adding a dehydrating agent such as, but not limited to, acetic anhydride, trifluoroacetic anhydride, polyphosphoric acid, phosphorus pentaoxide, phosphorus pentachloride, or thionyl chloride, or a mixture thereof. A catalyst, such as pyridine, can be used. In a thermal imidization method, the polyamic acid solution obtained in the above reaction is subjected to dehydration by mixing a solvent, such as benzene, toluene, xylene, mesitylene, chlorobenzene, diglyme, or triglyme, in an arbitrary amount into the polyamic acid solution and heating the resultant mixture while removing water formed by ring closure from the reaction system. These solvents can be used individually or in combination.

Next, a thermosetting resin (c) having a crosslinkable group is described. For improving the adhesive properties, heat resistance, and the like, it is preferred that the resin composition of the present invention comprises, in addition to the above-obtained polyimide (a) and/or polyamic acid (a′) and compound (b) of the general formula (I) of the present invention, a thermosetting resin (c) having a crosslinkable group. As component (c), especially, it is preferable to use an imide oligomer and/or isoimide oligomer having a crosslinkable group represented by the following general formulas (21) to (24):

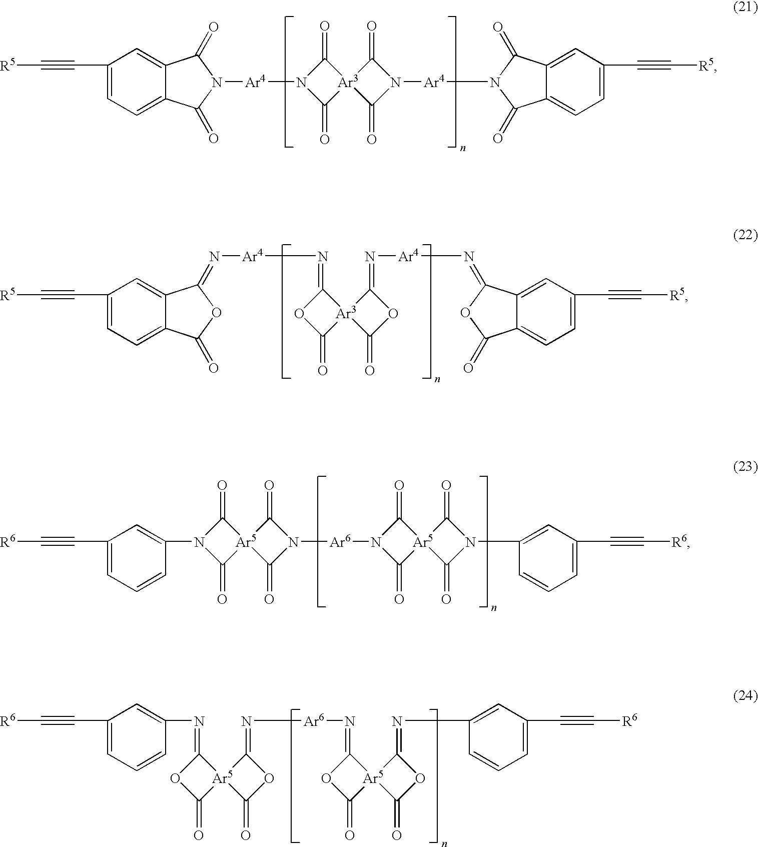

wherein n represents a number of 0 to 20; each of R⁵ and R⁶ independently represents hydrogen, 2-hydroxy-2-propyl, or a phenyl group; each of Ar³ and Ar⁵ independently represents a tetracarboxylic acid residue having 6 to 36 carbon atoms; and each of Ar⁴ and Ar⁶ independently represents a diamine residue having 6 to 36 carbon atoms.

In the preparation method for the imide oligomer or isoimide oligomer having a crosslinkable group, a corresponding amic acid oligomer is first prepared. With respect to the preparation of an amic acid oligomer, there is no particular limitation, and the preparation can be conducted by a known method, and is generally conducted in a solvent. The amic acid oligomer is prepared by the reaction of an aromatic tetracarboxylic dianhydride, an aromatic diamine, and an amine or acid molecular-end capping agent having a crosslinkable group in a polar solvent. Specific examples of the tetracarboxylic dianhydrides used in the preparation include pyromellitic dianhydride, 3,3′,4,4′-biphenyltetracarboxylic dianhydride, 2,3′,3,4′-biphenyltetracarboxylic dianhydride, 2,2′,3,3′-biphenyltetracarboxylic dianhydride, 4,4′-oxydiphthalic dianhydride, 3,4′-oxydiphthalic dianhydride, 3,3′-oxydiphthalic dianhydride, 3,3′,4,4′-benzophenonetetracarboxylic dianhydride, 3,3′,4,4′-diphenyl sulfone tetracarboxylic dianhydride, 2,2-bis(3,4-dicarboxyphenyl)propane dianhydride, 2,2-bis(3,4-dicarboxyphenyl)hexafluoropropane dianhydride, and 1,2,7,8-naphthalenetetracarboxylic dianhydride.

From the viewpoint of obtaining a resin having excellent flowability, it is desired that the imide oligomer and/or isoimide oligomer has a glass transition temperature of 250° C. or lower, especially 200° C. or lower. In the present invention, a glass transition temperature is defined as the temperature measured by a differential scanning calorimeter (hereinafter, referred to as “DSC”). From the viewpoint of easy availability of the raw material compounds in addition to the desired glass transition temperature, the tetracarboxylic dianhydride used varies depending on the type of the diamine compound used or the desired molecular weight, but it is desirable to use pyromellitic dianhydride, 4,4′-oxydiphthalic dianhydride, 3,3′,4,4′-biphenyltetracarboxylic dianhydride, 3,3′,4,4′-benzophenonetetracarboxylic dianhydride, 3,3′,4,4′-diphenyl sulfone tetracarboxylic dianhydride, 2,2-bis(3,4-dicarboxyphenyl)propane dianhydride, or 2,2-bis(3,4-dicarboxyphenyl)hexafluoropropane dianhydride. The above two or more dianhydrides can be used in combination.

Examples of aromatic diamines include aromatic diamines having one aromatic group, such as p-phenylenediamine, m-phenylenediamine, p-aminobenzylamine, m-aminobenzylamine, diaminotoluenes, diaminoxylenes, diaminonaphthalenes, and diaminoanthracenes; aromatic diamines having two aromatic groups, such as 4,4′-diaminobiphenyl, 3,4′-diaminobiphenyl, 3,3′-diaminobiphenyl, o-tolidine, m-tolidine, o-dianisidine, 4,4′-diaminodiphenylmethane, 3,4′-diaminodiphenylmethane, 3,3′-diaminodiphenylmethane, 4,4′-diaminodiphenyl ether, 3,4′-diaminodiphenyl ether, 3,3′-diaminodiphenyl ether, 4,4′-diaminodiphenyl sulfone, 3,4′-diaminodiphenyl sulfone, 3,3′-diaminodiphenyl sulfone, 4,4′-diaminodiphenyl ketone, 3,4′-diaminodiphenyl ketone, 3,3′-diaminodiphenyl ketone, 2,2-bis(4-aminophenoxy)propane, 2,2-bis(3-aminophenoxy)propane, and 2-(3-aminophenyl)-2-(4-aminophenyl)propane; aromatic diamines having three aromatic groups, such as 1,4-bis(4-aminophenoxy)benzene, 1,4-bis(3-aminophenoxy)benzene, 1,3-bis(4-aminophenoxy)benzene, 1,3-bis(3-aminophenoxy)benzene, 1,4-bis(4-aminobenzoyl)benzene, 1,4-bis(3-aminobenzoyl)benzene, 1,3-bis(4-aminobenzoyl)benzene, 1,3-bis(3-aminobenzoyl)benzene, and 9,9-bis(4-aminophenyl)fluorene; aromatic diamines having four or more aromatic groups, such as 2,2-bis[4-(4-aminophenoxy)phenyl]propane, 4,4′-bis(4-aminophenoxy)biphenyl, 4,4′-bis(3-aminophenoxy)biphenyl, bis[4-(4-aminophenoxy)phenyl]sulfone, bis[4-(3-aminophenoxy)phenyl]sulfone, bis[4-(4-aminophenoxy)phenyl]ether, bis[4-(3-aminophenoxy)phenyl]ether, 4,4′-bis(4-aminophenoxy)benzophenone, 4,4′-bis(3-aminophenoxy)benzophenone, 1,4-bis[4-(2-,3-, or 4-aminophenoxy)benzoyl]benzene, 1,3-bis[4-(2-,3-, or 4-aminophenoxy)benzoyl]benzene, 1,4-bis[3-(2-,3-, or 4-aminophenoxy)benzoyl]benzene, 1,3-bis[3-(2-,3-, or 4-aminophenoxy)benzoyl]benzene, 4,4′-bis[4-(2-,3-, or 4-aminophenoxy)benzoyl]diphenyl ether, 4,4′-bis[3-(2-,3-, or 4-aminophenoxy)benzoyl]diphenyl ether, 4,4′-bis[4-(2-, 3-, or 4-aminophenoxy)benzoyl]diphenyl, 4,4′-bis[3-(2-,3-, or 4-aminophenoxy)benzoyl]biphenyl, 4,4′-bis[4-(2-,3-, or 4-aminophenoxy)benzoyl]diphenyl sulfone, and 4,4′-bis[3-(2-, 3-, or 4-aminophenoxy)benzoyl]diphenyl sulfone. From the fact that the imide oligomer and/or isoimide oligomer has a glass transition temperature of 250° C. or lower, desirably 200° C. or lower from the viewpoint of obtaining a resin having excellent flowability, and from the viewpoint of easy availability, the aromatic diamine used varies depending on the type of the tetracarboxylic dianhydride used or the desired molecular weight, but, specifically, it is preferable to use p-phenylenediamine, m-phenylenediamine, 4,4′-diaminodiphenyl ether, 3,4′-diaminodiphenyl ether, 1,3-bis(4-aminophenoxy)benzene, 1,3-bis(3-aminophenoxy)benzene, 2,2-bis[4-(4-aminophenoxy)phenyl]propane, 4,4′-bis(4-aminophenoxy)biphenyl, 4,4′-bis(3-aminophenoxy)biphenyl, bis[4-(4-aminophenoxy)phenyl]sulfone, bis[4-(3-aminophenoxy)phenyl]sulfone, or 9,9-bis(4-aminophenyl)fluorene. The above two or more diamine compounds can be used in combination.

Examples of molecular-end capping agents having a crosslinkable group include acid molecular-end capping agents, such as 4-ethynylphthalic anhydride, 3-ethynylphthalic anhydride, 4-phenylethynylphthalic anhydride, 3-phenylethynylphthalic anhydride, 4-(3-hydroxy-3-methyl-1-but-1-ynyl)phthalic anhydride, 4-(3-hydroxy-3-methyl-1-but-1-ynyl)phthalic anhydride, ethynylnaphthalenedicarboxylic anhydride, phenylethynylnaphthalenedicarboxylic anhydride, ethynylanthracenedicarboxylic anhydride, phenylethynylanthracenedicarboxylic anhydride, 4-naphthylethynylphthalic anhydride, 3-naphthylethynylphthalic anhydride, naphthylethynylnaphthalenedicarboxylic anhydride, naphthylethynylanthracenedicarboxylic anhydride, 4-anthracenylethynylphthalic anhydride, 3-anthracenylethynylphthalic anhydride, anthracenylethynylnaphthalenedicarboxylic anhydride, and anthracenylethynylanthracenedicarboxylic anhydride. In these anhydrides, the hydrogen atom on the aromatic ring can be replaced by an alkyl group, alkenyl group, alkynyl group or alkoxy group having 1 to 6 carbon atoms, or a halogen atom. From the viewpoint of easy availability, it is desirable to use 4-phenylethynylphthalic anhydride, 4-ethynylphthalic anhydride, 4-(3-hydroxy-3-methyl-1-but-1-ynyl)phthalic anhydride or the like. The above two or more anhydrides can be used in combination.

Specific examples of amine molecular-end capping agents include 3-aminophenylacetylene, 4-aminophenylacetylene, 3-phenylethynylaniline, 4-phenylethynylaniline, 4-(3-hydroxy-3-methyl-1-but-1-ynyl)aniline, 4-(3-hydroxy-3-methyl-1-but-1-ynyl)aniline, 3-naphthylethynylaniline, 4-naphthylethynylaniline, 3-anthracenylethynylaniline, and 4-anthracenylethynylaniline. In these amines, the hydrogen atom on the aromatic ring can be replaced by an alkyl group, alkenyl group, alkynyl group or alkoxy group having 1 to 6 carbon atoms, or a halogen atom. From the viewpoint of easy availability, it is desirable to use 3-aminophenylacetylene, 4-aminophenylacetylene, 3-phenylethynylaniline, 4-phenylethynylaniline, or 4-(3-hydroxy-3-methyl-1-but-1-ynyl)aniline. The above two or more dianhydrides can be used in combination.

The desired molecular weight of the imide oligomer or isoimide oligomer depends on the molecular weight of the amic acid oligomer which is a precursor of the imide or isoimide oligomer.

The amount of the molecular-end capping agent having a crosslinkable group added varies depending on the desired molecular weight of the amic acid oligomer, but the amount is generally a mole 1 to several times, desirably 1.5 to 4 times the difference in mole between the tetracarboxylic dianhydride and the diamine compound. When the mole of the tetracarboxylic dianhydride is larger than one of the diamine compound, the amine molecular-end capping agent is used, and, when the mole of the diamine compound is larger than one of the dianhydride, an acid molecular-end capping agent is used.

With respect to the solvent used in the preparation of an amic acid oligomer, there is no particular limitation as long as it is an inert solvent in the reaction, and, for example, N,N-dimethylformamide, N,N-dimethylacetamide, N-methyl-2-pyrrolidone, dimethyl sulfoxide, tetramethylurea, tetrahydrofuran or the like can be used individually or in the form of a mixed solvent. Especially preferred is N,N-dimethylacetamide or N-methyl-2-pyrrolidone. A solvent, such as benzene, toluene, xylene, mesitylene, chlorobenzene, diglyme, or triglyme, can be mixed into the above solvent in an arbitrary amount. The reaction is generally conducted at a solute concentration of 5 to 80%.

Next, the imidization and isoimidization of an amic acid oligomer are described. The imidization reaction is conducted by dehydrating the amic acid oligomer obtained in the above reaction by a known method. For example, in a chemical imidization method, the amic acid oligomer solution obtained in the above reaction is subjected to dehydration by adding a dehydrating agent such as, but not limited to, acetic anhydride, trifluoroacetic anhydride, polyphosphoric acid, phosphorus pentaoxide, phosphorus pentachloride, or thionyl chloride, or a mixture thereof. A catalyst, such as pyridine, can be used. In a thermal imidization method, the amic acid oligomer solution obtained in the above reaction is subjected to dehydration by mixing a solvent, such as benzene, toluene, xylene, mesitylene, chlorobenzene, diglyme, or triglyme, in an arbitrary amount into the amic acid oligomer solution and heating the resultant mixture while removing water formed by ring closure from the reaction system. These solvents can be used individually or in combination. The isoimidization reaction is conducted by dehydrating the amic acid oligomer obtained in the above reaction by a known method. For example, the amic acid oligomer is subjected to dehydration by adding a dehydrating agent, such as trifluoroacetic anhydride or N,N-dicyclohexylcarbodiimide, or a mixture thereof. A catalyst, such as pyridine, can be used.

The imide oligomer or isoimide oligomer in the present invention can be used either in the form of powder obtained by pouring the reaction mixture obtained after the imidization or isoimidization into a solvent, such as water or alcohol, to effect reprecipitation, and collecting the resultant crystals by filtration and drying them, or in the form of a solution obtained merely by removing by-products of an isoimidizing agent, such as dicyclohexylurea, by filtration from the reaction mixture.

With respect to the resin composition of the present invention, it is preferred that the compound of general formula (I) of the present invention is mixed as a reactive monomer into the resin composition comprising the above-obtained polyimide (a) or polyamic acid (a′) and optionally imide oligomer and/or isoimide oligomer (c) having a crosslinkable group in a weight ratio of 99/1 to 40/60, desirably 95/5 to 50/50 (in terms of a solids content), and the resin composition can be obtained in the form of varnish or powder.

The heat-resistant adhesive agent of the present invention can be prepared from the resin composition of the present invention in the form of varnish or powder. With respect to the solvent used in the preparation of the heat-resistant adhesive agent, there is no particular limitation as long as the solvent has no chemical reactivity with each component and each component is soluble in the solvent. The solvent is appropriately selected from the solvents used in the preparation of the varnish mentioned above, or from lower alcohols (such as methanol, ethanol, propanol, isopropanol, and butanol), lower alkanes (such as pentane, hexane, heptane, and cyclohexane), ketones (such as acetone, methyl ethyl ketone, and methyl isobutyl ketone), halogenated hydrocarbon (such as dichloromethane, carbon tetrachloride, and fluorobenzene), aromatic hydrocarbons (such as benzene, toluene, and xylene), esters (such as methyl acetate, ethyl acetate, and butyl acetate) or the like, which can be used individually or in the form of a mixed solvent. With respect to the concentration of the resin composition of the present invention in the heat-resistant adhesive agent, there is no particular limitation, and the concentration is appropriately selected depending on the solubility of each component or the usage pattern of the heat-resistant adhesive agent, and, for example, a solute concentration of 5 to 80% is preferred. Further, filler or an additive can be added in such an amount that the effect of the present invention is not sacrificed.

Similarly, the varnish of the present invention can be prepared from the resin composition of the present invention in the form of varnish or powder. With respect to the solvent used in the preparation of the varnish, there is no particular limitation as long as each component is soluble in the solvent, and the reaction solvent used in the preparation of the individual components is preferably used. As the solvent, for example, N,N-dimethylformamide, N,N-dimethylacetamide, N-methyl-2-pyrrolidone, dimethyl sulfoxide, tetramethylurea, tetrahydrofuran or the like can be used individually or in the form of a mixed solvent. Especially preferred is N,N-dimethylacetamide, N-methyl-2-pyrrolidone, or tetrahydrofuran. A solvent, such as benzene, toluene, xylene, mesitylene, chlorobenzene, diglyme, or triglyme, can be mixed into the above solvent in an arbitrary amount. Solutions obtained by subjecting the reaction mixtures obtained after the reactions for the individual components to appropriate post-treatment can be mixed to prepare a varnish. With respect to the concentration of the resin composition of the present invention in the varnish, there is no particular limitation, and the concentration is appropriately selected depending on the solubility of each component or the form of the varnish used, and, for example, the solute concentration is 5 to 80%.

A film can be produced from the resin composition of the present invention. Generally, a varnish comprising the resin composition of the present invention is applied to a substrate comprised of, e.g., glass, aluminum, copper, stainless steel, a PET film, or a polyimide film, and dried to remove the solvent, obtaining a film having a desired thickness, preferably a thickness of 1 to 200 μm, more preferably 1 to 100 μm. If desired, the film obtained is subjected to appropriate curing treatment at 180 to 450° C. to obtain a cured product.

Next, the metal laminate of the present invention is described. The metal laminate of the present invention comprises an aromatic polymer as an insulating layer and a metallic foil as a conductive layer, such as a copper foil, which are laminated through the heat-resistant adhesive agent of the present invention. The aromatic polymer in the present invention may be any aromatic polymer having at least one benzene ring in the repeating units of the backbone and having insulation properties, and examples include polyimide, polysulfone, polyphenylene sulfide, polyaryl ether ketone, polycarbonate, liquid crystalline polymers, polybenzoxazole and the like.

In the preparation method for the metal laminate of the present invention, for example, a laminate composed of an aromatic polymer or a metallic foil and the heat-resistant adhesive agent of the present invention is first prepared. A varnish is applied to an aromatic polymer having a thickness of 1 to 200 μm, desirably 5 to 100 μm, further desirably 10 to 75 μm, or a metallic foil such as a copper foil as a conductive layer, so that the thickness of the above-mentioned heat-resistant adhesive agent can be 0.1 to 100 μm, desirably 1 to 30 μm, further desirably 1 to 10 μm after removing the solvent from the varnish, and then the solvent is removed by drying. The laminate composed of the aromatic polymer or metallic foil/heat-resistant adhesive agent is obtained and then, a metallic foil or an aromatic polymer is further stacked on the laminate by heat lamination to obtain a laminate composed of insulating layer/adhesive agent layer/conductive layer. The heat-resistant adhesive agent of the present invention has extremely excellent adhesive properties to an aromatic polymer or a metallic foil such that it does not need a surface treatment which has conventionally been made for improving the adhesive properties, such as a chemical treatment, sandblasting, or a plasma treatment. For the purpose of improving the wettability of the aromatic polymer surface and preventing the occurrence of cissing in the applied film of the heat-resistant adhesive agent to obtain a film having a uniform thickness, the above surface treatment can be conducted. A plasma treatment is especially preferred for obtaining a uniform film thickness.

The metallic foil, especially preferably a copper foil has a thickness of 0.1 to 100 μm, desirably 0.5 to 36 μm, further desirably 1 to 18 μm. When it is too thick, it is difficult to process the fine wiring having a line/space of 25 μm/25 μm or smaller, and, when it is too thin, it is difficult to handle it during the lamination.

The temperature for heat lamination is 100 to 300° C., desirably 120 to 250° C., further desirably 120 to 200° C. When the lamination temperature is higher than 300° C., wrinkles possibly occur in the metal laminate prepared due to a difference in dimensional change rate between the metallic foil, heat-resistant bonding agent, and aromatic polymer, thus producing defective products having bad appearance, poor insulation, poor conduction or the like. Further, oxidation of the metal is inevitable.

For example, when a very thin copper foil (0.1 to 5 μm) and an aromatic polymer are laminated together, a very thin copper foil attached to a PET film support is used. Generally, the usable temperature range of a PET film is 190° C. or lower, but the lamination using a general thermoplastic polyimide adhesive agent requires a temperature of 250° C. or higher, and PET suffers marked heat shrinkage at such a high temperature, causing warpage. In addition, a problem occurs in that the PET film is melted to cause pollution of the apparatus. In contrast, the use of the heat-resistant adhesive agent of the present invention enables lamination at 190° C. or lower, namely, enables lamination of a copper foil attached to a PET film support, thus facilitating the preparation of a very thin copper foil laminate.

The varnish is applied to at least one surface of an aromatic polymer film so that the thickness of the heat-resistant adhesive agent of the present invention can be 0.1 to 100 μm, desirably 1 to 30 μm, further desirably 1 to 10 μm after removing the solvent from the varnish, and then dried to remove the solvent to obtain an aromatic polymer/heat-resistant adhesive agent laminate. Another aromatic polymer film is stacked on and stuck to the laminate obtained, or the film-form aromatic polymer/heat-resistant adhesive agent laminate is rolled into a cylindrical form and stuck, thus obtaining an aromatic polymer laminate or a cylindrical aromatic polymer.

The thus obtained metal laminate or aromatic polymer laminate is subjected to heat treatment at 200 to 450° C., desirably 250 to 400° C., for 10 seconds to 60 minutes, desirably 1 to 10 minutes to further promote curing of the heat-resistant adhesive agent used in the metal laminate or aromatic polymer laminate, making it possible to further improve the heat resistance. As a heat treatment oven for the heat treatment, an arbitrary heat treatment oven, such as a vacuum dryer, a hot-air dryer, or a far-infrared oven, can be used. Especially in the heat treatment for the metal laminate, for preventing the oxidation of metal, it is desired that the heat treatment is conducted in a vacuum or in an inert atmosphere.

With respect to the metal laminate of the present invention which has been cured, a peel strength between the metallic foil and the aromatic polymer is 0.5 kN/m or more, desirably 0.8 kN/m or more, further desirably 1.0 kN/m or more. When the peel strength is low, problems such as peeling or blistering occur during the step for circuit processing or COF mounting.

When the reactive monomer represented by the general formula (I) in the present invention is thermally cured solely or, if necessary, together with an additive, such as an epoxy resin, an acrylic resin, filler, reinforcing fibers, a release agent, or a colorant, the resultant cured product can be used as, for example, a molding material, an encapsulation material for use in a semiconductor package, a coating material, or a prepreg. Specifically, the curing can be conducted by a heat treatment in an organic solvent or in the absence of a solvent at a temperature of 100 to 400° C., more desirably 200 to 380° C. under atmospheric pressure or under a pressure using a molding machine or the like for about 10 minutes to 12 hours, more desirably about 30 minutes to 4 hours. For example, in a semiconductor package, an encapsulation material obtained from the reactive monomer represented by the general formula (I) of the present invention can be used as a molding resin, and molded and cured to encapsulate a semiconductor device.

### EXAMPLES

Hereinbelow, the embodiment of the present invention will be described in more detail with reference to the following Examples and Comparative Examples, which should not be construed as limiting the scope of the present invention.

In the following Examples, the methods for measuring purity, melting point or glass transition temperature, NMR, infrared absorption spectrum, and peel strength are as follows.

### Purity:

1 mg of the compound was dissolved in 1 mL of tetrahydrofuran (THF), and a purity of the solution was measured by liquid chromatography (LC-10AD; manufactured by Shimadzu Corporation) under conditions such that the column was TSK gel ODS-80™ (manufactured by Tosoh Corp.), the column temperature was 40° C., the mobile phase was THF/H₂O=550/450, the flow rate was 1.0 mL/min, and the detector was UV 254 nm.

### Melting Point or Glass Transition Temperature:

A measurement was conducted by means of a differential scanning calorimeter (DSC-60; manufactured by Shimadzu Corporation) by making temperature elevation of from 40 to 400° C. at a rate of 5° C. per minute. A melting point or glass transition temperature was determined from the extrapolated point of a DSC curve by making a calculation using an analysis software.

### NMR:

A solution was prepared by mixing the compound and deuterated DMSO (DMSO-d₆ containing 0.05% TMS; manufactured by Cambrige Isotope Laboratories, Inc.), and subjected to ¹H-NMR measurement by NMR (JNM-AL400; manufactured by JEOL LTD.).

### Infrared Absorption Spectrum:

An IR absorption spectrum was measured by a KBr tablet method by means of an IR spectrometer (FTIR-8200; manufactured by Shimadzu Corporation).

### Peel Strength of Metal Laminate:

A metal was etched to 1-mm width using an aqueous solution of ferric chloride, and then the aromatic polymer side was stuck to a stainless steel plate having a thickness of 1 mm using a double-sided adhesive tape, and the metal was peeled in the 180° direction at a speed of 50 mm/min using a tensile tester (Autograph AGS-H; manufactured by Shimadzu Corporation) to determine a peel strength.

### Peel Strength of Aromatic Polymer Laminate:

An aromatic polymer laminate was cut into a 10-mm width, and the aromatic polymer on one side was stuck to a stainless steel plate having a thickness of 1 mm using a double-sided adhesive tape, and the aromatic polymer on the other side was peeled in the 180° direction at a speed of 50 mm/min using a tensile tester (Autograph AGS-H; manufactured by Shimadzu Corporation) to determine a peel strength.

### Example 1

### Synthesis of N-(3-ethynylphenyl)-4′-phenylethynylphthalimide

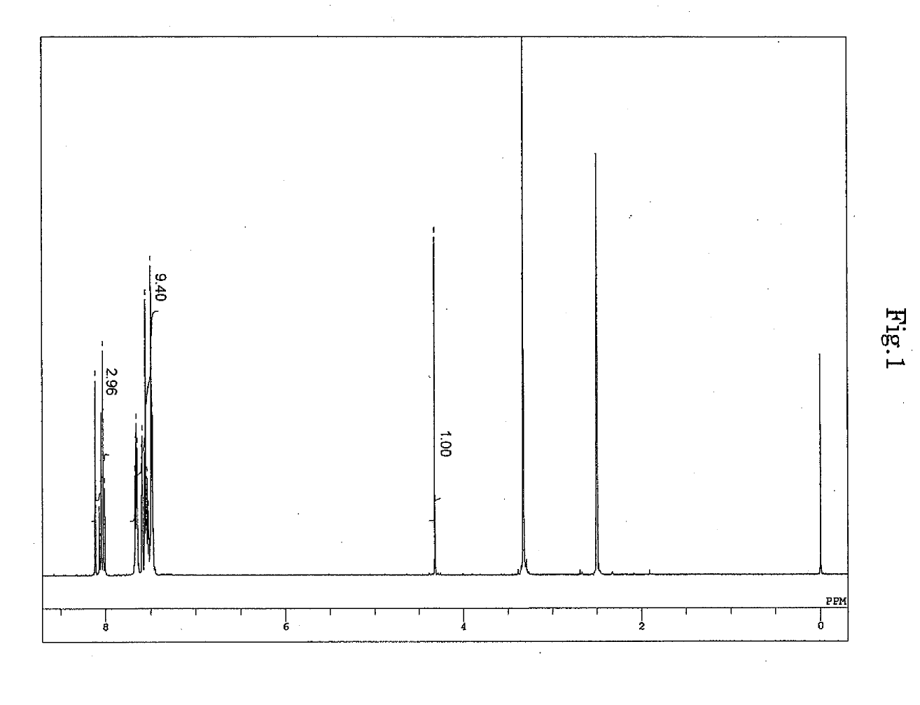

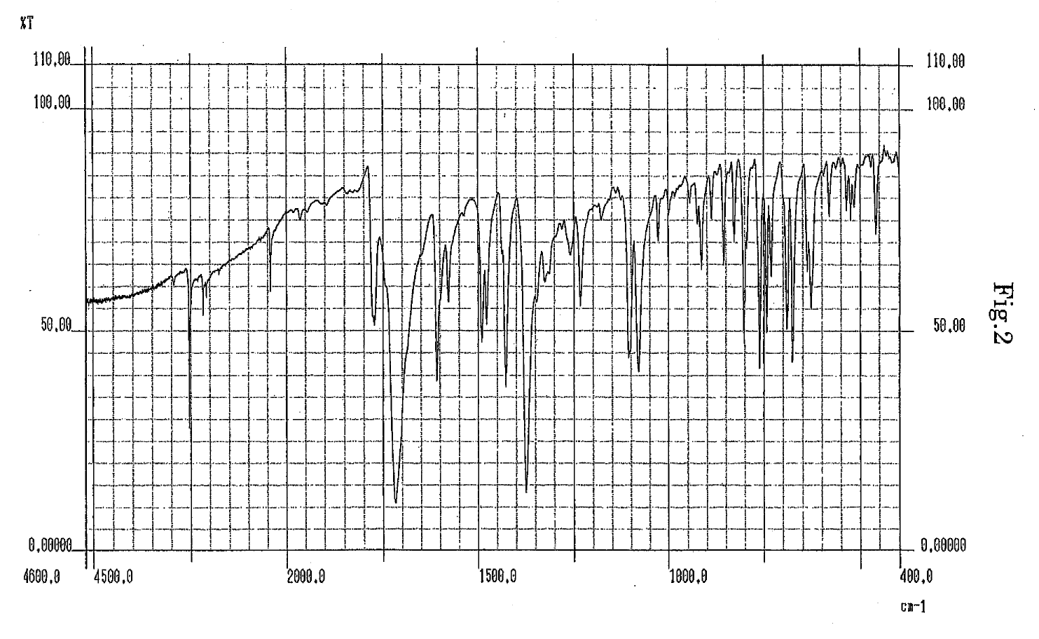

Into a four-necked flask were charged 23.4296 g (0.20 mol) of 3-aminophenylacetylene, 414.1 g of N-methyl-2-pyrrolidone, and 41.4 g of xylene, and the solids were dissolved under a nitrogen gas stream. 49.6466 g (0.20 mol) of 4-phenylethynylphthalic anhydride was added in portions to the resultant solution and stirred at room temperature for 4 hours to form a yellow amic acid solution. Subsequently, the flask was heated to 200° C. under reflux for 8 hours while removing water formed by the imidization together with xylene from the reaction system. The resultant reaction mixture was cooled to room temperature so that crystals were precipitated, and the crystals were collected by filtration and dried to obtain crystals of N-(3-ethynylphenyl)-4′-phenylethynylphthalimide (yield: 70%; purity: 98%). The crystals obtained were subjected to DSC measurement. As a result, a melting point was observed at 212° C., and heat generation due to crosslinking of the triple bond was observed at temperature from 217° C. An NMR chart and an IR chart of the crystals are shown in FIG. 1 and FIG. 2, respectively.

### Example 2

### Synthesis of N-(3-ethynylphenyl)-4′-phenylethynylphthalisoimide

Into a four-necked flask were charged 23.4296 g (0.20 mol) of 3-aminophenylacetylene and 337.5 g of N-methyl-2-pyrrolidone, and the solids were dissolved under a nitrogen gas stream. 49.6466 g (0.20 mol) of 4-phenylethynylphthalic anhydride was added in portions to the resultant solution and stirred at room temperature for 4 hours to form a yellow auric acid solution. Subsequently, while cooling the flask to 5° C., a solution prepared by dissolving 41.3 g (0.20 mol) of dicyclohexylcarbodiimide (DCC) in 76.6 g of NMP was added dropwise to the flask from a dropping funnel over one hour. Then, the resultant mixture was warmed to room temperature and stirred for 3 hours, and then dicyclohexylurea (DCU) by-produced in the reaction was removed by filtration to obtain an isoimide solution having a solution concentration of 15% (yield: 90%; purity: 98%). A portion of the isoimide solution was poured into methanol so that crystals were precipitated, followed by filtration, to obtain crystals of the isoimide. The crystals obtained were subjected to DSC measurement. As a result, a melting point was observed at 191° C., and heat generation due to crosslinking of the triple bond was observed at temperature from 201° C.

### Example 3

### Synthesis of N-(3-ethynylphenyl)-4′-ethynylphthalimide

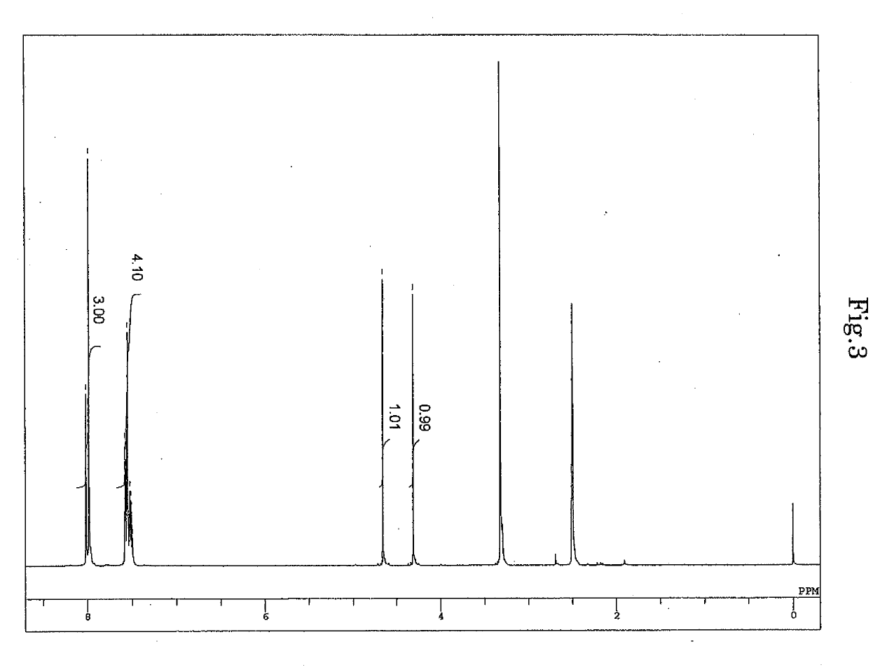

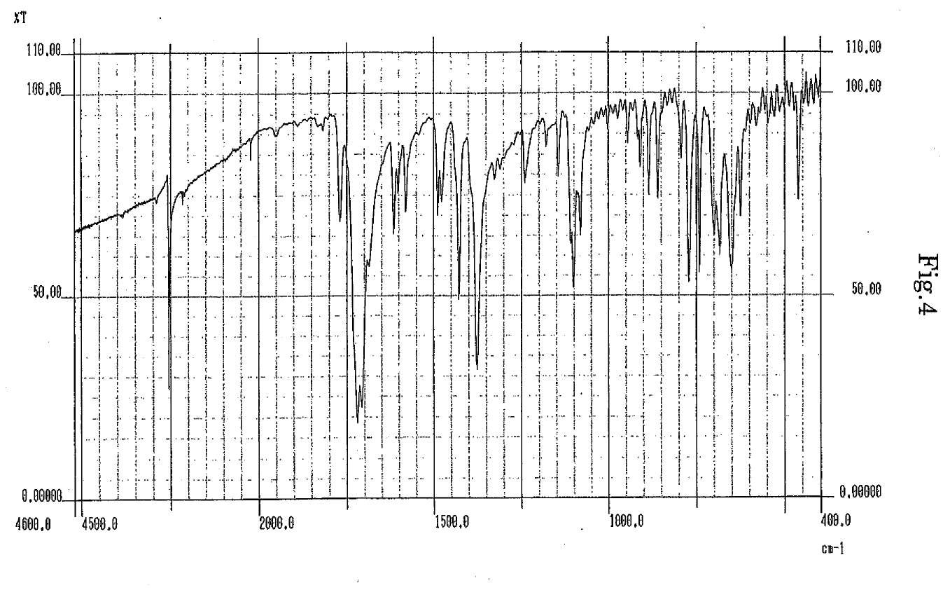

Into a four-necked flask were charged 23.4296 g (0.20 mol) of 3-aminophenylacetylene and 327.9 g of N-methyl-2-pyrrolidone, and the solids were dissolved under a nitrogen gas stream. 34.4274 g (0.20 mol) of 4-ethynylphthalic anhydride was added in portions to the resultant solution and stirred at room temperature for 4 hours to form a brown amic acid solution. Subsequently, 1.6 g (0.02 mol) of pyridine and 61.3 g (0.60 mol) of acetic anhydride were added to the solution from a dropping funnel. The resultant mixture was stirred at room temperature for 3 hours, and the precipitated crystals were collected by filtration and dried to obtain crystals of N-(3-ethynylphenyl)-4′-ethynylphthalimide. The crystals obtained were subjected to DSC measurement. As a result, heat generation due to crosslinking of the triple bond was observed at temperature from 220° C. An NMR chart and an IR chart of the crystals are shown in FIG. 3 and FIG. 4, respectively.

### Example 4

### Synthesis of N-[3-(3-hydroxy-3-methyl-1-but-1-ynyl)phenyl]-4′-phenylethynylphthalimide

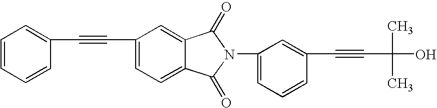

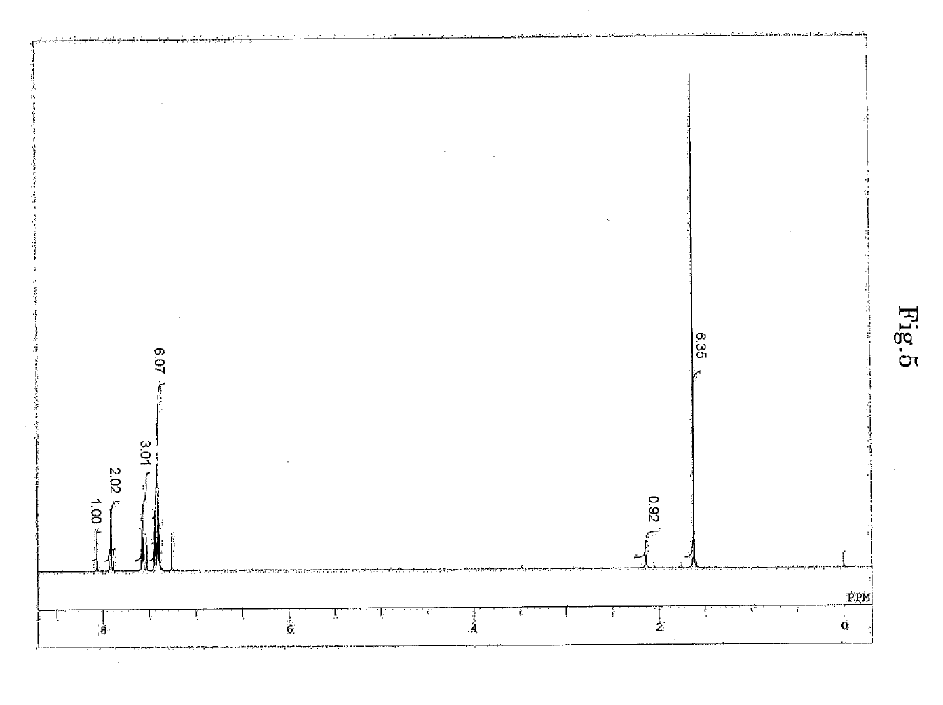

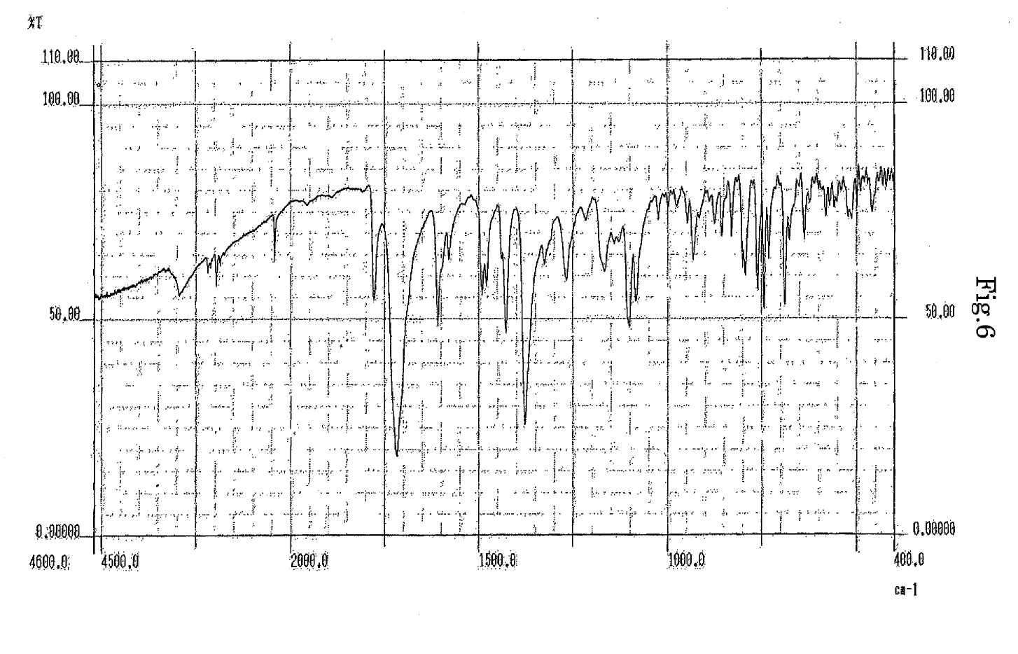

Into a four-necked flask were charged 17.5227 g (0.10 mol) of 4-(3-aminophenyl)-2-methyl-3-butyn-2-ol and 169.4 g of N-methyl-2-pyrrolidone, and the solids were dissolved under a nitrogen gas stream. 24.8233 g (0.10 mol) of 4-phenylethynylphthalic anhydride was added in portions to the resultant solution and stirred at room temperature for 4 hours to form a reddish brown amic acid solution. Subsequently, 0.8 g (0.01 mol) of pyridine and 30.7 g (0.30 mol) of acetic anhydride were added to the solution from a dropping funnel. The resultant mixture was stirred at room temperature for 3 hours, and poured into 2 L of water, and the precipitated crystals were collected by filtration and dried to obtain a desired compound. The crystals obtained were subjected to DSC measurement. As a result, a melting point was observed at 136° C., and heat generation due to crosslinking of the triple bond was observed at temperature from 269° C. An NMR chart and an IR chart of the crystals are shown in FIG. 5 and FIG. 6, respectively.

### Examples 5 to 7

The compounds shown below were individually synthesized in a similar manner as in Example 2 or 3 except that each component was changed. The results are shown in Table 1.

| | Example 5 | Example 6 | Example 7 |
|---|---|---|---|
| Compound | N-(4-Ethynylphenyl)-4′- | N-(4-Ethynylphenyl)- | N-(3-Ethynylphenyl)- |
| name | phenylethynylphthalimide | 4′-ethynylphthalimide | 4′- |
| | | | ethynylphthalisoimide |
| Acid | PEPA | EPA | EPA |
| component | | | |
| Amine | p-APA | p-APA | m-APA |
| component | | | |
| Ring closure | Acetic anhydride/ | Acetic anhydride/ | DCC |
| method | pyridine | pyridine | |
| Yield (%) | 79 | 55 | 90 |
| Purity (%) | 99 | 100 | 99 |
| Melting | 243 | Not observed | Not observed |
| point (° C.) | | | |
| Heat | 245 | 220 | 210 |
| generation | | | |
| (° C.) | | | |

Abbreviations shown in Table 1 indicate the following compounds.

PEPA: 4-Phenylethynylphthalic anhydride
EPA: 4-Ethynylphthalic anhydride
p-APA: p-Aminophenylacetylene
m-APA: m-Aminophenylacetylene

### DCC: N,N-Dicyclohexylcarbodiimide

### Synthesis Example 1

### Synthesis of Polyamic Acid

Into a four-necked flask were charged 21.8119 g (0.1 mol) of pyromellitic dianhydride, 16.0189 g (0.08 mol) of 4,4′-diaminodiphenyl ether, 2.1628 g (0.02 mol) of p-phenylenediamine, and 226.6 g of N-methyl-2-pyrrolidone (NMP), and the resultant mixture was stirred at room temperature for 4 hours to synthesize a polyamic acid. A polyamic acid solution having a solute concentration of 15% and a viscosity (Brookfield type viscometer; manufactured by Tokyo Keiki Co., Ltd.) of 10,000 mPa·s was obtained.

### Synthesis Example 2

### Synthesis of Polyimide

Into a four-necked flask were charged 12.4086 g (0.04 mol) of 4,4′-oxydiphthalic dianhydride, 16.4203 g (0.04 mol) of 2,2-bis[4-(4-aminophenoxy)phenyl]propane, 163.4 g of NMP, and 16.3 of xylene, and the resultant mixture was stirred under a nitrogen gas stream at room temperature for 2 hours to obtain a polyamic acid. Subsequently, the flask was heated to 200° C. under reflux for 8 hours while removing water formed by the imidization together with xylene from the reaction system. The resultant reaction mixture was cooled to room temperature to obtain a polyimide solution having a solute concentration of 15% and a viscosity of 80,000 mPa·s.

### Synthesis Example 3

### Synthesis of Isoimide Oligomer

Into a four-necked flask were charged 12.4086 g (0.04 mol) of 4,4′-oxydiphthalic dianhydride, 23.3866 g (0.08 mol) of 1,3-bis(3-aminophenoxy)benzene, 19.8568 g (0.08 mol) of 4-phenylethynylphthalic anhydride, and 254.0 g of NMP, and the resultant mixture was stirred under a nitrogen gas stream at room temperature for 3 hours. While cooling the flask to 5° C., a solution prepared by dissolving 33.0 g (0.16 mol) of dicyclohexylcarbodiimide (DCC) in 61.3 g of NMP was added dropwise to the flask from a dropping funnel over one hour. Then, the resultant mixture was warmed to room temperature and stirred for 3 hours, and then dicyclohexylurea (DCU) by-produced in the reaction was removed by filtration to obtain an isoimide oligomer solution having a solution concentration of 15%. A portion of the isoimide oligomer solution was poured into methanol so that crystals were precipitated, followed by filtration, to obtain crystals of the isoimide oligomer. The crystals obtained were subjected to DSC measurement. As a result, a glass transition temperature was observed at 98° C., heat generation due to rearrangement of the isoimide to imide was observed at temperature from 220° C., and heat generation due to crosslinking of the phenylethynyl group was observed at temperature from 365° C.

### Example 8

### Preparation of Adhesive Agent

The imide compound obtained in Example 1 was mixed into and dissolved in the polyamic acid solution obtained in Synthesis Example 1 in an amount of 15 wt %, based on the weight of the polyamic acid.

### Example 9

### Preparation of Adhesive Agent

The isoimide compound obtained in Example 2 was mixed into and dissolved in the polyimide solution obtained in Synthesis Example 2 in an amount of 15 wt %, based on the weight of the polyimide.

### Example 10

### Preparation of Adhesive Agent

The imide compound obtained in Example 4 was mixed into and dissolved in the polyamic acid solution obtained in Synthesis Example 1 in an amount of 15 wt %, based on the weight of the polyamic acid.

### Example 11

### Preparation of Adhesive Agent

The polyimide and the isoimide oligomer obtained in Synthesis Examples 2 and 3 were mixed in a solute weight ratio of 50:50, and the N-(3-ethynylphenyl)-4-phenylethynylphthalimide obtained in Example 1 was further mixed and dissolved in an amount of 10 wt %, based on the weight of the whole solids of the above varnish.

### Example 12

### Preparation of Polyimide Metal Laminate

The varnish obtained in Example 7 was applied to Kapton 200EN having a thickness of 50 μm so that the thickness of the adhesive agent layer is 2 μm after removing the solvent from the varnish, and then dried at 160° C. for 2 minutes to obtain a film sample. The film sample obtained and a copper foil having a thickness of 9 μm (CF-T8GD-SV; manufactured by FUKUDA METAL FOIL & POWDER Co., Ltd.) were stacked and subjected to lamination at a temperature of 175° C. The lamination could be achieved. The thus obtained metal laminate was cured in a vacuum at a temperature of 380° C. for 90 seconds, and subjected to peeling measurement. As a result, it was found that the adhesive force was 1.2 kN/m. The adhesive agent layer cured was subjected to DSC measurement. As a result, no glass transition temperature was observed.

### Example 13

### Preparation of Polyimide Metal Laminate

The varnish obtained in Example 8 was applied to Kapton 150EN having a thickness of 40 μm so that the thickness of the adhesive agent layer is 2 μm after removing the solvent from the varnish, and then dried at 160° C. for 2 minutes to obtain a film sample. The film sample obtained and a copper foil having a thickness of 9 μm (CF-T8GD-SV; manufactured by FUKUDA METAL FOIL & POWDER Co., Ltd.) were stacked and subjected to lamination at a temperature of 175° C. The lamination could be achieved. The thus obtained metal laminate was cured in a vacuum at a temperature of 380° C. for 90 seconds, and subjected to peeling measurement. As a result, it was found that the adhesive force was 1.1 kN/m. The adhesive agent layer cured was subjected to DCS measurement. As a result, it was found that the glass transition temperature was 285° C.

### Example 14

### Preparation of Polyimide Metal Laminate

The varnish obtained in Example 9 was applied to Kapton 150EN having a thickness of 40 pin so that the thickness of the adhesive agent layer is 2 μm after removing the solvent from the varnish, and then dried at 160° C. for 2 minutes to obtain a film sample. The film sample obtained and a copper foil having a thickness of 9 μm (CF-T8GD-SV; manufactured by FUKUDA METAL FOIL & POWDER Co., Ltd.) were stacked and subjected to lamination at a temperature of 170° C. The lamination could be achieved. The thus obtained metal laminate was cured in a vacuum at a temperature of 380° C. for 90 seconds, and subjected to peel strength measurement. As a result, it was found that the adhesive force was 1.5 kN/m. The adhesive agent layer cured was subjected to DSC measurement. As a result, no glass transition temperature was observed.

### Example 15

### Preparation of Polyimide Metal Laminate

The varnish obtained in Example 9 was applied to Kapton 200EN having a thickness of 50 μm so that the thickness of the adhesive agent layer is 3 μm after removing the solvent from the varnish, and then dried at 160° C. for 2 minutes to obtain a film sample. The film sample obtained and a copper foil having a thickness of 9 pin (CF-T8GD-SV; manufactured by FUKUDA METAL FOIL & POWDER Co., Ltd.) were stacked and subjected to lamination at a temperature of 160° C. The lamination could be achieved. The thus obtained metal laminate was cured in a vacuum at a temperature of 380° C. for 90 seconds, and subjected to peeling measurement. As a result, it was found that the adhesive force was 1.8 kN/m. The adhesive agent layer cured was subjected to DSC measurement. As a result, it was found that the glass transition temperature was 295° C.

### Example 16

Lamination was conducted in a similar manner as in Example 15 except that the copper foil used was replaced by a copper foil attached to a separate film and having a thickness of 1.5 μm (CKPF-5CQ; manufactured by FUKUDA METAL FOIL & POWDER Co., Ltd.). The lamination could be achieved at 175° C., and the cured laminate exhibited peel strength, i.e., adhesive force of 1.7 kN/m.

### Example 17

### Preparation of Polyimide Laminate

Lamination was conducted in a similar manner as in Example 12 except that Kapton 200EN having a thickness of 50 μm was used instead of the copper foil. The lamination could be achieved at 175° C., and the cured laminate exhibited peel strength, i.e., adhesive force of 1.5 kN/m.

### Comparative Example 1

A polyimide bonding film containing no N-(3-ethynylphenyl)-4-phenylethynylphthalimide was prepared using the polyamic acid solution obtained in Synthesis Example 1. Lamination was conducted in a similar manner as in Example 10. The lamination could not be achieved at 175° C.

### Comparative Example 2

A polyimide adhesive film containing no N-(3-ethynylphenyl)-4-phenylethynylphthalimide was prepared using a varnish obtained by mixing the polyimide and the isoimide oligomer obtained in Synthesis Examples 2 and 3 in a solute weight ratio of 50:50. Lamination was attempted using various copper foils at various temperatures, but the lamination could not be achieved at a temperature lower than 265° C.

### INDUSTRIAL APPLICABILITY

The resin composition comprising the compound of general formula (I) of the present invention as a reactive monomer and the heat-resistant adhesive agent obtained from the resin composition individually have excellent melting properties and excellent flowability at a relatively low temperature, and have excellent adhesive properties with respect to a metallic foil at a low temperature. Further, they can be used in lamination with a very thin copper foil attached to a PET film support, and a cured product obtained by crosslinking or curing the above resin composition or adhesive agent by a heat treatment has excellent adhesive properties and excellent resistance to soldering heat as well as excellent' electrical properties, and is especially preferably used in the production of a metal laminate for use in COF mounting which needs fine wiring.

1. A compound represented by the following general formula (I): 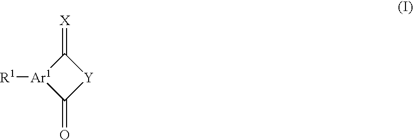
wherein:
one of X and Y represents O and the other represents NAr²R²;
R¹ and R², which may be the same or different, represent hydrogen or an organic group having 2 to 36 carbon atoms and containing at least one carbon-carbon double bond or carbon-carbon triple bond, with the proviso that R¹ and R² are not simultaneously hydrogen;
Ar¹ represents an organic group having 6 to 36 carbon atoms; and
Ar² represents an organic group having 6 to 36 carbon atoms.

2. The compound of general formula (I) according to claim 1, wherein R¹ is a group represented by the following formula (3): 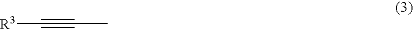
wherein R³ represents hydrogen or an organic group having 1 to 34 carbon atoms.

3. The compound of general formula (I) according to claim 1, wherein R² is a group represented by the following formula (4): 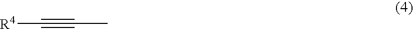
wherein R⁴ represents hydrogen or an organic group having 1 to 34 carbon atoms.

4. The compound of general formula (I) according to claim 2, wherein R³ is hydrogen, a C₆-C₁₈ aryl group, or a group represented by the following formula: 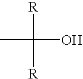
wherein each R independently represents hydrogen, a C₁-C₄ alkyl group, or a C₆-C₁₈ aryl group.

5. The compound of general formula (I) according to claim 2, wherein R³ is hydrogen, a phenyl group, or a group represented by the following formula:

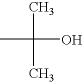

6. The compound of general formula (I) according to claim 3, wherein R⁴ is hydrogen, a C₆-C₁₈ aryl group, or a group represented by the following formula: 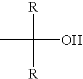
wherein each R independently represents hydrogen, a C₁-C₄ alkyl group, or a C₆-C₁₈ aryl group.

7. The compound of general formula (I) according to claim 3, wherein R⁴ is hydrogen, a phenyl group, or a group represented by the following formula:

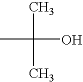

8. The compound of general formula (I) according to claim 1, wherein R¹ and R², which may be the same or different, are selected from ethynyl, phenylethynyl, and a group represented by the following formula:

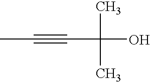

9. The compound of general formula (I) according to claim 1, wherein Ar¹ is benzenetriyl and Ar² is phenylene.

10. The compound of general formula (I) according to claim 1, which is selected from compounds of the following formulas (5) to (12):

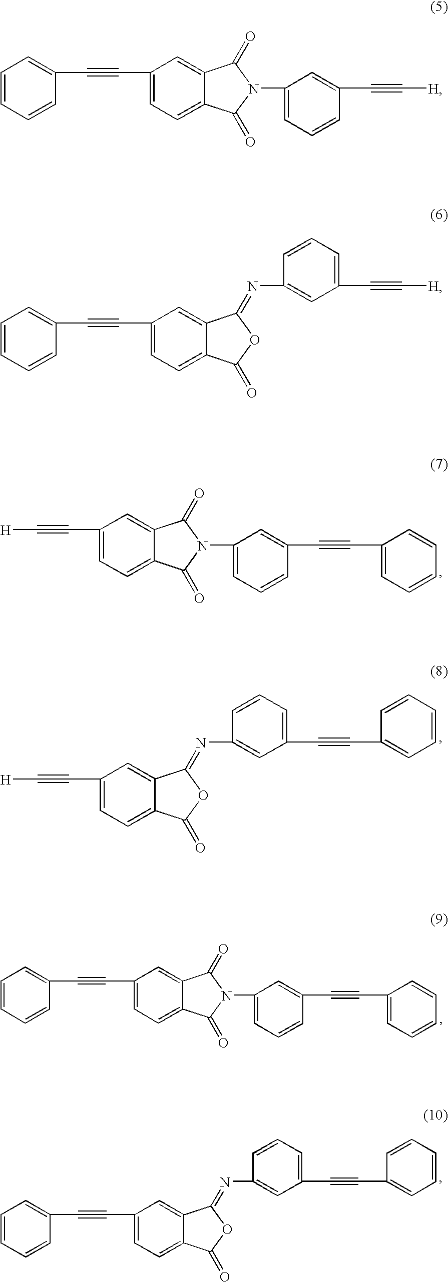

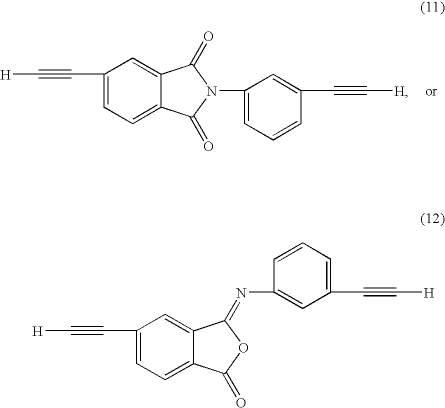

11. The compound of general formula (I) according to claim 1, which is selected from compounds of the following formulas (13) to (17):

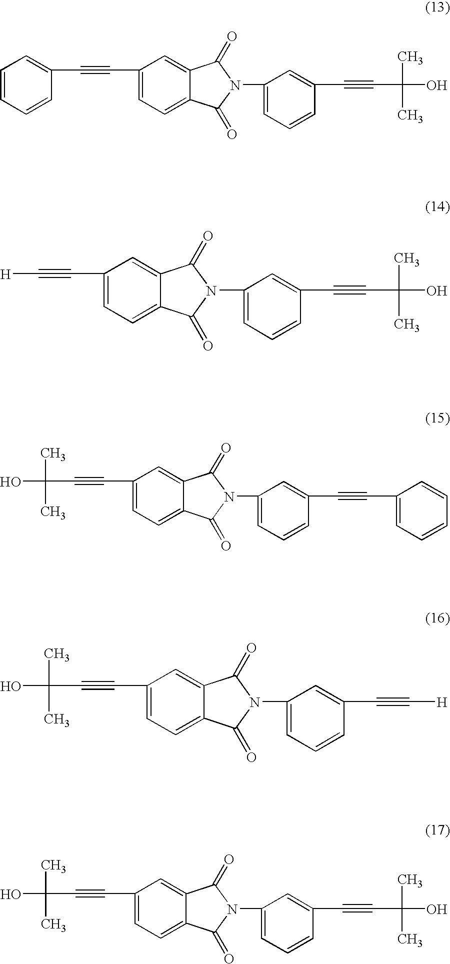

12. A resin composition comprising:
(a) polyimide; and
(b) the compound of general formula (I) according to claim 1.

13. A resin composition comprising:
(a′) polyamic acid; and
(b) the compound of general formula (I) according to any one of claim 1.

14. The resin composition according to claim 12, which contains the polyimide (a) or polyamic acid (a′) and the compound (b) of general formula (I) according to claim 1 in a weight ratio of 99/1 to 40/60.

15. A resin composition comprising:
(a) polyimide;
(b) the compound of general formula (I) according to claim 1; and
(c) a thermosetting resin having a crosslinkable group.

16. A resin composition comprising:
(a′) polyamic acid;
(b) the compound of general formula (I) according to claim 1; and
(c) a thermosetting resin having a crosslinkable group.

17. The resin composition according to claim 15, wherein the thermosetting resin (c) having a crosslinkable group is selected from the following general formulas (21) to (24): 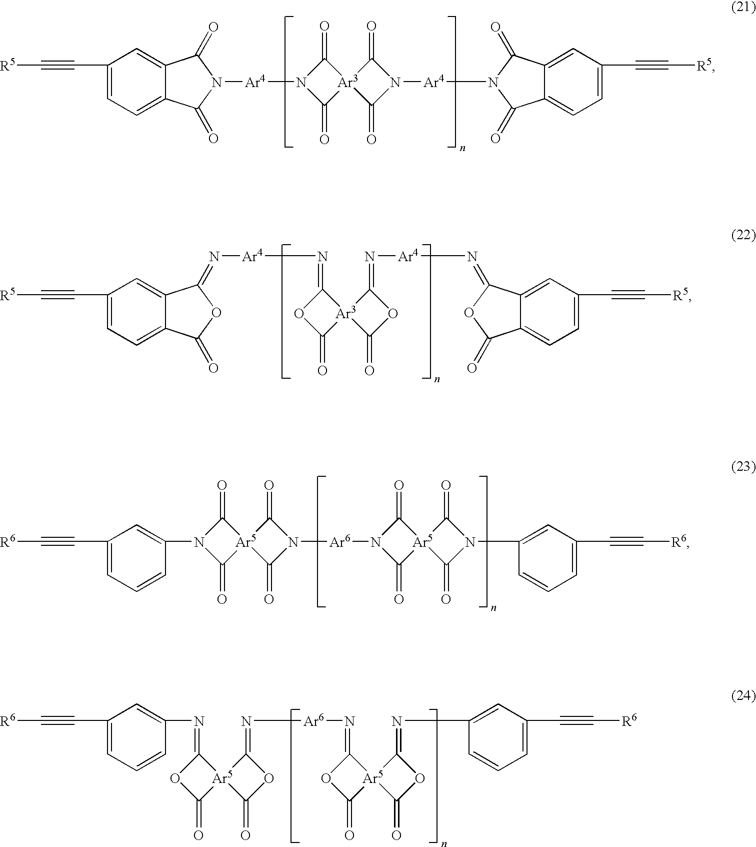
wherein n represents a number of 0 to 20; R⁵ and R⁶ independently represent hydrogen, 2-hydroxy-2-propyl, or a phenyl group; Ar³ and Ar⁵ independently represent a tetracarboxylic acid residue having 6 to 36 carbon atoms; and Ar⁴ and Ar⁶ independently represent a diamine residue having 6 to 36 carbon atoms.

18. The resin composition according to claim 15, which contains the polyimide (a) or polyamic acid (a′) and the thermosetting resin (c) having a crosslinkable group in a weight ratio of 95/5 to 5/95.

19. The resin composition according to claim 15, wherein the ratio of the total weight of the polyimide (a) or polyamic acid (a′) and the thermosetting resin (c) having a crosslinkable group to the weight of the compound (b) of general formula (I) according to claim 1 is 99/1 to 40/60.

20. The resin composition according to claim 15, wherein the thermosetting resin (c) having a crosslinkable group has a glass transition temperature of 200° C. or lower.

21. A heat-resistant adhesive agent comprising the resin composition according to claim 12.

22. A varnish comprising the resin composition according to claim 12.

23. A film obtained by applying the varnish according to claim 22 to a substrate and drying it.

24. A metal laminate comprising an insulating layer comprised of an aromatic polymer having on at least one surface thereof a metallic foil laminated through the heat-resistant adhesive agent according to claim 21.

25. The metal laminate according to claim 24, wherein the metallic foil has a thickness of 0.1 to 18 μm.

26. The metal laminate according to claim 24, wherein the aromatic polymer is selected from polyimide, polysulfone, polyphenylene sulfide, polyaryl ether ketone, polycarbonate, a liquid crystalline polymer, and polybenzoxazole.

27. The metal laminate according to claim 24, wherein the aromatic polymer has a plasma-treated surface.

28. An electronic circuit using the metal laminate according to claim 24.

29. An aromatic polymer laminate or cylindrical aromatic polymer comprising an aromatic polymer film having on at least one surface thereof another aromatic polymer film laminated through the heat-resistant adhesive agent according to claim 21.

30. A cured product obtained by thermally curing the compound of general formula (I) according to claim 1.
